(19) United States
(12) Patent Application Publication
(10) Pub. No.: US 2023/0355513 A1
(43) Pub. Date: Nov. 9, 2023

(54) PARTICLES INCLUDING CHITOSAN-BILIRUBIN CONJUGATE, AND ORAL PHARMACEUTICAL COMPOSITION INCLUDING SAME

(71) Applicant: KOREA ADVANCED INSTITUTE OF SCIENCE AND TECHNOLOGY, Daejeon (KR)

(72) Inventors: Sangyong JON, Daejeon (KR); Afia Tasnim RAHMAN, Daejeon (KR); Chang-Hee WHANG, Daejeon (KR)

(21) Appl. No.: 18/219,956

(22) Filed: Jul. 10, 2023

Related U.S. Application Data
(63) Continuation-in-part of application No. PCT/KR2022/000686, filed on Jan. 13, 2022.

(30) Foreign Application Priority Data
Jan. 13, 2021 (KR) 10-2021-0004953

(51) Int. Cl. A61K 9/00; A61P 1/00; C08B 37/08; A61K 31/409; A61K 47/69; A61P 29/00

(52) U.S. Cl. CPC A61K 9/0053; A61P 1/00; C08B 37/003; A61K 31/409; A61K 47/6939; A61P 29/00

(57) ABSTRACT

The present disclosure relates to: a chitosan-bilirubin conjugate; particles; and an oral pharmaceutical composition including same. The conjugate and/or particles according to the present disclosure have excellent antioxidant and anti-inflammatory effects, exhibit systemic inflammation relieving effects as well as intestinal inflammatory reaction relieving effects, and have the effect of normalizing the balance of intestinal microflora distribution, and thus, can be useful for treating inflammatory bowel diseases, systemic and chronic inflammatory diseases, etc.

### CROSS-REFERENCE TO RELATED APPLICATIONS

This application is a continuation in part of PCT Application No. PCT/KR2022/000686 filed on Jan. 13, 2022, which claims the benefit and priority to Korean Patent Application No. 10-2021-0004953, filed on Jan. 13, 2021. The entire disclosure of the applications identified in this paragraph are incorporated herein by references.

### FIELD

The present disclosure was made with the support of the Ministry of Science and ICT, Republic of Korea, under project identification No. 1711111754, project No. 2018R1A3B1052661, which was conducted in the research project named “Development of Tumor Microenvironment Targeting and Sensitive Precision Bio-Nanomedicine Research Center” in the research program titled “Basic Research Project in Field of Science and Engineering”, by the Korea Advanced Institute of Science and Technology, under management of the National Research Foundation of Korea, from 1 Mar. 2020 to 28 Feb. 2021.

The present disclosure relates to particles containing a chitosan-bilirubin conjugate and a pharmaceutical composition containing the same.

### BACKGROUND

Inflammatory bowel disease appears in many people worldwide, and the development of medicines therefor is being actively studied. However, most medicines focus simply on reducing inflammation, and their effects in the intestinal function normalization after inflammation are still insufficiently studied.

In the normal intestine, intestinal functions are regulated by continuous interactions between intestinal epithelial cells and intestinal microbes. Intestinal microbes contribute to inhibiting major pathways so that unnecessary inflammatory responses are not made under normal conditions, leading to the overall intestinal microbial balance and the mucus layer maintenance (Nature 448, 427-434 (2007)). When an inflammatory response occurs in the intestine, the balance of intestinal microbes is disrupted by inflammatory substances and an excessive amount of reactive oxygen species is generated, and thus the intestinal wall collapses and the mucous layer is difficult to maintain. Moreover, large amounts of immune cells are driven to cause more severe inflammation. Therefore, in order to reduce the inflammatory response in the intestine, the delivery of antioxidants capable of scavenging reactive oxygen species or the like is needed. If antioxidative effects suppress inflammation as well as normalize the intestinal microflora, the antioxidants are expected to be able to act much more effectively in the recovery of intestinal functions.

According to previous studies, bilirubin is well known to have an anti-inflammatory effect through its strong antioxidative action. However, bilirubin has very low hydrophilicity, and thus bilirubin alone cannot be used as a drug, and particularly, bilirubin is difficult to deliver by oral administration.

[Reference Document]

### Non-Patent Document

R. J. Xavier et al., Nature 448, 427-434 (2007)

### SUMMARY

### Technical Problem

The present inventors endeavored to improve properties of bilirubin to enable bilirubin to be well dissolved in water by conjugation of bilirubin with hydrophilic chitosan so that bilirubin having strong antioxidative action can be orally administered. The resultant prepared chitosan-bilirubin conjugate was confirmed to form nanoparticles by self-assembly in an aqueous solvent and to exhibit excellent antioxidative and anti-inflammatory effects when administered orally. Furthermore, the conjugate and particles were confirmed to have effects of alleviating the intestinal inflammatory response as well as systemic inflammation and be also effective in regulating the balance of intestinal microbes.

Accordingly, an aspect of the present disclosure is to provide a conjugate of hydrophilic chitosan and bilirubin.

Another aspect of the present disclosure is to provide particles containing the conjugate.

Still another aspect of the present disclosure is to provide a pharmaceutical composition for anti-inflammation containing: the conjugate, the particles, or a combination thereof; and a pharmaceutically acceptable carrier.

Still another aspect of the present disclosure is to provide a method for the treatment of an inflammatory disease, the method including administering the pharmaceutical composition for anti-inflammation to a subject in need of treatment.

### Solution to Problem

In accordance with an aspect of the present disclosure, there is provided a conjugate containing a hydrophilic chitosan and bilirubin, the hydrophilic chitosan being linked to bilirubin.

Herein, chitosan is a kind of polysaccharide obtained from chitin and has both biocompatibility and biodegradability. However, chito-oligosaccharides obtained by deacetylation from chitin have relatively poor affinity for water. Therefore, the present inventors prepared chitosan having a shorter length, that is, low-molecular weight chitosan, from a long chain, through an additional process. Specifically, low-molecular weight chitosan (LMWC) was prepared through fractionation of commercially produced chito-oligosaccharides.

Herein, “conjugate of hydrophilic chitosan and bilirubin” according to an aspect of the present disclosure may be expressed as (hydrophilic or low-molecular weight) chitosan-bilirubin, a (hydrophilic or low-molecular weight) chitosan-bilirubin conjugate, a (hydrophilic or low-molecular weight) chitosan and bilirubin conjugate, LMWC-BR, an LMWC-BR conjugate, a conjugate, or the like. The above parentheses mean that the expression within the parentheses can be omitted.

Herein, the term expressing the above-described chitosan-bilirubin conjugate is also used as a term meaning particles manufactured from the chitosan-bilirubin conjugate with respect to embodiments of the present disclosure. Specifically, the term “particles” manufactured from the chitosan-bilirubin conjugate refers to particles produced by self-assembly when the chitosan-bilirubin conjugate is dissolved in an aqueous solvent. The hydrodynamic diameter, measured by DLS, of the particles may be 10 to 5,000 nm, more specifically, 10 to 4,000 nm, 10 to 3,000 nm, 10 to 2,000 nm, 10 to 1,000 nm, 10 to 800 nm, 10 to 600 nm, 10 to 500 nm, 10 to 400 nm, 10 to 350 nm, 10 to 300 nm, 10 to 250 nm, 10 to 220 nm, 10 to 200 nm, 10 to 150 nm, 10 to 140 nm, 10 to 130 nm, 10 to 120 nm, or 10 to 110 nm, but is not limited thereto. The particles of the present disclosure have a size in micro to nano units as described above. Therefore, the particles of the present disclosure are expressed as microparticles or nanoparticles depending on the diameter of the particles.

In an embodiment of the present disclosure, the hydrophilic chitosan is linked to bilirubin via a covalent bond.

In an embodiment of the present disclosure, the hydrophilic chitosan is linked to bilirubin via an amide linkage. More specifically, the conjugate is obtained by an amide linkage between a carboxyl group of bilirubin and an amine group of the hydrophilic chitosan.

In an embodiment of the present disclosure, the carboxyl group of bilirubin is activated by a carboxyl group activator for a conjugation reaction (e.g., amide linkage reaction). Examples of the carboxyl group activator is 1-ethyl-3-(3-dimethylaminopropyl)carbodiimide (EDC), dicyclohexylcarbodiimide (DCC), or N,N′-diisopropylcarbodiimide (DIC), but are not limited thereto. In a specific embodiment of the present disclosure, the carboxyl group activator is EDC.

In the present disclosure, the carboxyl group of bilirubin reacts with EDC to form an active O-acylisourea intermediate, which is then displaced by nucleophilic attack from the primary amine group of chitosan in the reaction mixture. If desired, N-hydroxysulfosuccinimide (Sulfo-NHS) is added during the reaction of chitosan with the primary amine. With the sulfo-NHS addition, the EDC couples NHS to the carboxyl group, forming an NHS ester that is more stable than the O-acylisourea intermediate while allowing for efficient conjugation to the primary amine at physiologic pH. In either event, the result is a covalent bond between bilirubin and chitosan. Other chemical reactions, such as Suzuki-Miyaura cross-coupling, succinimidyl 4-(N-maleimido methyl) cyclohexane-1-carboxylate (SMCC), or aldehyde based reactions may alternatively be used.

In an embodiment of the present disclosure, the hydrophilic chitosan has a molecular weight of 3 kDa to 30 kDa, more specifically, 3 kDa to 25 kDa, 3 kDa to 20 kDa, 3 kDa to 15 kDa, 3 kDa to 12 kDa, 3 kDa to 10 kDa, 3 kDa to 5 kDa, 5 kDa to 30 kDa, 5 kDa to 25 kDa, 5 kDa to 20 kDa, 5 kDa to 15 kDa, 5 kDa to 12 kDa, 5 kDa to 10 kDa, 7 kDa to 30 kDa, 7 kDa to 25 kDa, 7 kDa to 20 kDa, 7 kDa to 15 kDa, 7 kDa to 12 kDa, 7 kDa to 10 kDa, 8 kDa to 30 kDa, 8 kDa to 25 kDa, 8 kDa to 20 kDa, 8 kDa to 15 kDa, 8 kDa to 12 kDa, 8 kDa to 10 kDa, 10 kDa to 30 kDa, 10 kDa to 25 kDa, 10 kDa to 20 kDa, 10 kDa to 15 kDa, 10 kDa to 12 kDa, 12 kDa to 30 kDa, 12 kDa to 25 kDa, 12 kDa to 20 kDa, 12 kDa to 15 kDa, 3 kDa, 5 kDa, 7 kDa, 8 kDa, 10 kDa, 12 kDa, 15 kDa, 20 kDa, 25 kDa, or 30 kDa, but is not limited thereto.

In a specific embodiment of the present disclosure, the conjugate has a structure of Formula 1 below:

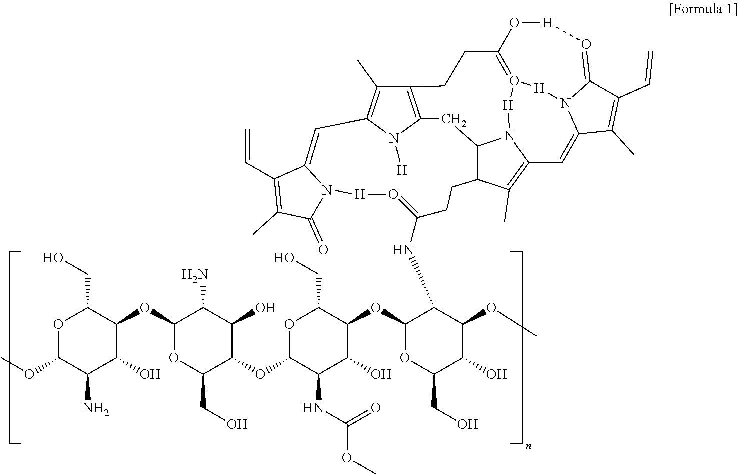

where n is an integer of 4 to 45, more specifically, 4-40, 4-35, 4-30, 4-25, 4-20, 4-15, 4-14, 4-13, 4-12, 4-11, 4-10, 4-9, 4-8, 5-45, 5-40, 5-35, 5-30, 5-25, 5-20, 5-15, 5-14, 5-13, 5-12, 5-11, 5-10, 5-9, 5-8, 6-45, 6-40, 6-35, 6-30, 6-25, 6-20, 6-15, 6-14, 6-13, 6-12, 6-11, 6-10, 6-9, 6-8, 7-45, 7-40, 7-35, 7-30, 7-25, 7-20, 7-15, 7-14, 7-13, 7-12, 7-11, 7-10, 7-9, 7-8, 8-45, 8-40, 8-35, 8-30, 8-25, 8-20, 8-15, 8-14, 8-13, 8-12, 8-11, 8-10, 8-9, 9-45, 9-40, 9-35, 9-30, 9-25, 9-20, 9-15, 9-14, 9-13, 9-12, 9-11, 9-10, 10-45, 10-40, 10-35, 10-30, 10-25, 10-20, 10-15, 10-14, 10-13, 10-12, 10-11, 12-45, 12-40, 12-35, 12-30, 12-25, 12-20, 12-15, 12-14, 12-13, 14-45, 14-40, 14-35, 14-30, 14-25, 14-20, 14-15, 20-45, 20-40, 20-35, 20-30, 20-25, 25-45, 25-40, 25-35, 25-30, 30-45, 30-40, 30-35, or 40-45, or each integer corresponding to the above numerical ranges. In an embodiment of the present disclosure, the weight ratio of bilirubin and chitosan constituting the conjugate is 1:1 to 1:15, 1:1 to 1:10, 1:1 to 1:8, 1:1 to 1:7, 1:1 to 1:5, 1:1 to 1:4, 1:1 to 1:3, 1:1 to 1:2, 1:15, 1:10, 1:9, 1:8, 1:7, 1:6, 1:5, 1:4, 1:3, 1:2, or 1:1. The weight ratio includes integers between the above numerical ranges as well as numerical values corresponding to all decimal units.

In accordance with another aspect of the present disclosure, there are provided particles including a conjugate prepared by conjugation of the hydrophilic chitosan and bilirubin.

In an embodiment of the present disclosure, the particles are formed by self-assembly of a plurality of conjugates in an aqueous solution.

In the chitosan-bilirubin conjugate constituting the particles of the present disclosure, chitosan constitutes a hydrophilic moiety and bilirubin constitutes a hydrophobic moiety. The hydrophilic moiety means a hydrophilic polymer or small molecule, and the hydrophobic moiety means a hydrophobic polymer or small molecule. In the particles formed by self-assembly of the plurality of conjugates of the present disclosure, bilirubin constituting a hydrophobic moiety forms a micelle structure having a hydrophobic core, and chitosan constituting the hydrophilic moiety is oriented outside the core.

In an embodiment of the present disclosure, the hydrodynamic diameter of the particles, as measured by dynamic light scatting (DLS), is 10 to 5,000 nm, more specifically, 10 to 4,000 nm, 10 to 3,000 nm, 10 to 2,000 nm, 10 to 1,000 nm, 10 to 800 nm, 10 to 600 nm, 10 to 500 nm, 10 to 400 nm, 10 to 350 nm, 10 to 300 nm, 10 to 250 nm, 10 to 220 nm, 10 to 200 nm, 10 to 150 nm, 10 to 140 nm, 10 to 130 nm, 10 to 120 nm, or 10 to 110 nm; 20 to 350 nm, 20 to 220 nm, 20 to 200 nm, 20 to 150 nm, 20 to 140 nm, 20 to 130 nm, 20 to 120 nm, or 20 to 110 nm; 30 to 350 nm, 30 to 220 nm, 30 to 200 nm, 30 to 150 nm, 30 to 140 nm, 30 to 130 nm, 30 to 120 nm, or 30 to 110 nm; 40 to 350 nm, 40 to 220 nm, 40 to 200 nm, 40 to 150 nm, 40 to 140 nm, 40 to 130 nm, 40 to 120 nm, or 40 to 110 nm; 50 to 350 nm, 50 to 220 nm, 50 to 200 nm, 50 to 150 nm, 50 to 140 nm, 50 to 130 nm, 50 to 120 nm, or 50 to 110 nm; 100 to 350 nm, 100 to 220 nm, 100 to 200 nm, 100 to 150 nm, 100 to 140 nm, 100 to 130 nm, 100 to 120 nm, or 100 to 110 nm, but is not limited thereto.

In accordance with another aspect of the present disclosure, there is provided a pharmaceutical composition for anti-inflammation, the pharmaceutical composition containing: the chitosan-bilirubin conjugate, the particles, or a combination thereof; and a pharmaceutically acceptable carrier.

In an embodiment of the present disclosure, the pharmaceutical composition contains, as an active ingredient, particles manufactured of the chitosan-bilirubin conjugate.

In an embodiment of the present disclosure, the particles of the present disclosure have excellent ability to scavenge ROS, such as H₂O₂, AAPH, and NaOCl, and thus can be used as an anti-oxidative agent. Specifically, the particles of the present disclosure, which are orally administered into the body, can target an inflammatory site by an enhanced permeability and retention (EPR) effect. At the inflammation site, the nanoparticles can exhibit anti-inflammatory activity by scavenging an abnormal level of reactive oxygen species.

In an embodiment of the present disclosure, the particles of the present disclosure target to act on macrophages. More specifically, the particles of the present disclosure inhibit the expression and secretion of cytokines involved in inflammation, such as IL-1beta, IL-6, and TNF-alpha, in macrophages, and stimulates expression and secretion of cytokines involved in the restoration of damaged tissues, such as TGF-beta and IL-10, and thus can be helpfully used as an anti-inflammatory agent or an inflammation reducing agent.

In an embodiment of the present disclosure, the particles of the present disclosure exhibit an effect of normalizing the weight loss caused by an inflammatory bowel disease.

In an embodiment of the present disclosure, the particles of the present disclosure exhibit an effect of lowering the disease activity index (DAI) caused by an inflammatory bowel disease.

In an embodiment of the present disclosure, the particles of the present disclosure exhibit an effect of normalizing the decrease in colon length caused by an inflammatory bowel disease.

In an embodiment of the present disclosure, the particles of the present disclosure prevent or treat atrophy of the spleen, which is a symptom of systemic inflammation caused by inflammatory bowel disease.

In an embodiment of the present disclosure, the particles of the present disclosure exhibit an effect of lowering blood ALT and blood AST, which are numerical indicators indicating hepatitis caused by inflammatory bowel disease, to close to normal levels.

In an embodiment of the present disclosure, the particles of the present disclosure exhibit an effect of lowering blood creatine and blood BUN, which are numerical indicators of kidney inflammation or kidney function caused by inflammatory bowel disease, to close to normal levels.

In an embodiment of the present disclosure, the particles of the present disclosure exhibit an effect of lowering the levels of blood IL-6 and blood TNF-alpha, which are numerical indicators indicating the presence or absence of systemic inflammation caused by inflammatory bowel disease, to close to normal levels.

In an embodiment of the present disclosure, the particles of the present disclosure exhibit an effect of increasing the mRNA expression levels of ZO-1, Claudin-1, and Occludin-1 genes, which are indicators indicating the intestinal damage by inflammatory bowel disease, to close to normal levels.

Furthermore, in an embodiment of the present disclosure, the particles of the present disclosure have an unpredictably excellent effect, with respect to the microbiome distribution, to normalize the microbiome distribution of the inflammatory bowel disease model mice to the microbiome distribution similar to that of normal mice.

The pharmaceutical composition of the present disclosure is formulated using a pharmaceutically acceptable carrier and/or excipient according to the method that is easily conducted by a person having ordinary skills in the art to which the present disclosure pertains, and the composition of the present disclosure may be prepared into a unit dosage form or may be inserted into a multi-dose container. The formulation may be a solution, a suspension, or an emulsion in oil or aqueous medium, and may further contain a dispersant or a stabilizer.

When the composition of the present disclosure is a pharmaceutical composition, the pharmaceutically acceptable carrier is a one that is conventionally used in the formulation, and examples thereof may include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, and a mineral oil, but are not limited thereto. The pharmaceutical composition of the present disclosure may further contain, in addition to the above ingredients, a lubricant, a wetting agent, a sweetening agent, a flavoring agent, an emulsifier, a suspending agent, a preservative, and the like.

In an embodiment of the present disclosure, the pharmaceutical composition of the present disclosure may further contain an excipient of sucrose, mannitol, sorbitol, glycerin, trehalose, or polyethylene glycol, and an excipient of cyclodextrin (alpha-, beta-, and gamma-cyclodextrin, hydroxy cyclodextrin, or a cyclodextrin derivative). The excipient is added to the particles, which correspond to an active ingredient of the present pharmaceutical composition, serves as a cryoprotectant or an osmoregulator, and is formulated by freeze-drying, solvent evaporation, or the like.

The pharmaceutical composition of the present disclosure can be orally and parenterally. For example, the pharmaceutical composition of the present disclosure may be administered intraperitoneally, intramuscularly, subcutaneously or topically. In addition, intrarectal administration, inhalation administration, intranasal administration, or the like can be employed. In an embodiment of the present disclosure, the pharmaceutical composition is used for oral administration.

In an embodiment of the present disclosure, the chitosan-bilirubin conjugate or the nanoparticles formed by self-assembly thereof of the present disclosure contains a low-molecular weight chitosan exhibiting intestinal adhesion and thus is appropriate for, especially, (formulations) for oral administration.

The appropriate dose of the pharmaceutical composition of the present disclosure varies depending on factors, such as formulation method, administration method, patient's age, body weight, and sex, pathological condition, diet, administration time, administration route, excretion rate, and response sensitivity, and an ordinarily skilled practitioner can easily determine and prescribe a dose that is effective for desired treatment or prevention. According to a preferable embodiment of the present disclosure, the daily dose of the pharmaceutical composition of the present disclosure is 0.001-100 mg/kg.

As used herein, the term “administration” refers to providing a predetermined substance for a subject in any appropriate manner. The administration route of the composition of the present disclosure may encompass all the general routes, and as described above, the composition may be administered orally or parenterally. In addition, the composition of the present disclosure may be administered by using any apparatus that can deliver an active ingredient to a target cell, tissue, or organ.

As used herein, the term “subject” refers to, but is not particularly limited to, for example, a human, monkey, cow, horse, sheep, pig, chicken, turkey, quail, cat, dog, mouse, rat, rabbit, or guinea pig, preferably a mammal, and more preferably a human.

In an embodiment of the present disclosure, the anti-inflammation means use for prevention and treatment of an inflammatory disease. Examples of the inflammatory disease includes inflammatory bowel disease (IBD), atopic dermatitis, edema, dermatitis, allergies, asthma, conjunctivitis, periodontitis, rhinitis, otitis media, atherosclerosis, pharyngolaryngitis, tonsillitis, pneumonia, gastric ulcers, gastritis, Crohn's disease, colitis, hemorrhoids, gout, ankylosing spondylitis, rheumatic fever, lupus, fibromyalgia, psoriatic arthritis, osteoarthritis, rheumatoid arthritis, Periarthritis of shoulder, tendinitis, tenosynovitis, myositis, hepatitis, cystitis, nephritis, Sjogren's syndrome, multiple sclerosis, and the like.

In an embodiment of the present disclosure, the anti-inflammation means use for prevention and treatment of a chronic inflammatory disease. Examples of the chronic inflammatory disease include non-alcoholic steatohepatitis, pneumonia, pulmonary fibrosis, nephritis, kidney failure, cystitis, Sjogren's syndrome, multiple sclerosis, asthma, atherosclerosis, myocardial infarction, pancreatitis, diabetes, psoriasis, osteoporosis, arthritis, osteoarthritis, rheumatoid arthritis, systemic inflammatory syndrome, sepsis, dementia, and the like. The chronic inflammatory disease may be caused due to a systemic inflammatory response, or cause an inflammatory response all over the body by the occurrence of the inflammatory disease. As used herein, the term “inflammatory bowel disease” refers to a disease in which inflammation occurs in the intestine, that is, the small intestine and the large intestine, and includes a disease in which abnormal chronic inflammation in the intestine repeats remission and recurrence. The inflammatory bowel disease includes specific enteritis with known causes, non-specific enteritis with unknown causes, and enteritis caused from other diseases, for example, intestinal Behcet's disease.

In one embodiment of the present disclosure, the inflammatory bowel disease is selected from the group consisting of ulcerative colitis, Crohn's disease, intestinal Behcet's disease, indeterminate colitis, bacterial enteritis, viral enteritis, amoebic enteritis, hemorrhagic rectal ulcer, leaky gut syndrome, ischemic colitis, and tuberculous enteritis, but is not limited thereto. More specifically, the inflammatory bow disease is ulcerative colitis or Crohn's disease.

According to an embodiment of the present disclosure, the chitosan-bilirubin conjugate or the particles containing the same of the present disclosure, which are an active ingredient of the pharmaceutical composition of the present disclosure, has an effect of improving indicators showing systemic inflammation as well as inflammation in the intestine when administered orally, and thus can be helpfully used as a pharmaceutical composition for the prevention or treatment of systemic and chronic inflammatory diseases.

As used herein, the term “prevention” refers to any action that inhibits or delays the symptoms of an inflammatory disease through the administration of the composition according to the present disclosure.

As used herein, the term “treatment” refers to any action that attains the remission or complete recovery of the symptoms of an inflammatory disease through the administration of the composition according to the present disclosure.

The pharmaceutical composition of the present disclosure contains a pharmaceutically effective amount of the particles of the present disclosure. The pharmaceutically effective amount refers to an amount that is sufficient for the particles to attain a pharmaceutical effect.

In addition, the pharmaceutical composition of the present disclosure may further contain an active ingredient that is known to have a treatment effect on an inflammatory bowel disease or a chronic inflammatory disease in the art. Examples of the active ingredient include steroids, such as glucocorticosteroid, 5-aminosalicylic acid (5-ASA)-based drugs, such as sulfasalazine and mesalazine, anti-TNF-α monoclonal antibodies, and the like.

In accordance with another aspect of the present disclosure, there is provided a food composition for inflammation alleviation, the food composition containing the chitosan-bilirubin conjugate, the particles, or a combination thereof.

In accordance with another aspect of the present disclosure, there is provided a food composition for anti-oxidation, the food composition containing the chitosan-bilirubin conjugate, the particles, or a combination thereof.

The food composition of the present disclosure may be prepared in the form of a powder, granules, a tablet, a capsule, a drink, or the like. Examples of the food composition include various foods such as candies, drinks, chewing gums, teas, vitamin complexes, health supplement foods, and the like.

The food composition of the present disclosure contains not only the chitosan-bilirubin conjugate, the particles, or a combination thereof, as an active ingredient, but also ingredients that are usually added in food manufacturing, for example, a protein, a carbohydrate, a fat, a nutrient, a seasoning agent, and a flavoring agent. Examples of the foregoing carbohydrate include: common sugars, such as monosaccharides (e.g., glucose and fructose), disaccharides (e.g., maltose, sucrose, and oligosaccharides), and polysaccharides (e.g., dextrin and cyclodextrin); and sugar alcohols, such as xylitol, sorbitol, and erythritol. As the flavoring agent, natural flavoring agents (thaumatin, stevia extracts (e.g., rebaudioside A, glycyrrhizin, etc.)) and synthetic flavoring agents (saccharin, aspartame, etc.) may be used. For example, when the food composition of the present disclosure is prepared as a drink, the drink may further contain citric acid, liquefied fructose, sugar, glucose, acetic acid, malic acid, fruit juice, an Eucommia ulmoides extract, a jujube extract, and a licorice extract, in addition to the chitosan-bilirubin conjugate, the particles, or a combination thereof of the present disclosure.

In accordance with still another aspect of the present disclosure, there is provided a feed composition for anti-inflammation or inflammation alleviation, the feed composition containing the chitosan-bilirubin conjugate, the particles, or a combination thereof.

Since the features of the chitosan-bilirubin conjugate, the particles, or a combination thereof contained in the food composition and the feed composition of the present disclosure overlap with those of the chitosan-bilirubin conjugate, the particles, or a combination thereof contained in the pharmaceutical composition of the present disclosure, the overlapping description therebetween is omitted in order to avoid excessive complexity of the present specification.

In accordance with another aspect of the present disclosure, there is provided a method for the treatment of an inflammatory disease, the method including administering the above-described pharmaceutical composition for anti-inflammation to a subject in need of treatment.

Since the method for treatment of an inflammatory disease contained in the food composition and the feed composition of the present disclosure overlap with those of the chitosan-bilirubin conjugate, the particles, or a combination thereof contained in the pharmaceutical composition of the present disclosure, the overlapping description therebetween is omitted in order to avoid excessive complexity of the present specification.

In accordance with another aspect of the present disclosure, there is provided a method for preparing a conjugate, the method including:
(a) reacting bilirubin with a carboxyl group activator to activate a carboxyl group of bilirubin; and
(b) reacting the carboxyl group of bilirubin with an amine group of a hydrophilic chitosan to form an amide linkage.

In an embodiment of the present disclosure, the carboxyl group activator is 1-ethyl-3-(3-dimethylaminopropyl)carbodiimide (EDC), dicyclohexylcarbodiimide (DCC), or N,N′-diisopropylcarbodiimide (DIC), but is not limited thereto.

In an embodiment of the present disclosure, in the reaction of step (a), N-hydroxysulfosuccinimide (Sulfo-NHS) is added.

### Advantageous Effects of Invention

The present disclosure provides a chitosan-bilirubin conjugate and particles containing the same.

Furthermore, the present disclosure provides a pharmaceutical composition containing the conjugate, the particles, or a combination thereof.

The pharmaceutical composition of the present disclosure containing the conjugate particles of the present disclosure has excellent antioxidative and anti-inflammatory effects, exhibits effects of alleviating intestinal inflammation responses as well as systemic inflammation, and has an effect of normalizing the balance of intestinal microbe distribution, and thus can be helpfully used in the treatment of inflammatory bowel disease, systemic or chronic inflammatory disease, or the like.

### BRIEF DESCRIPTION OF DRAWINGS

FIG. 1 is a schematic diagram of a low molecular weight chitosan production method.

FIG. 2A shows a reaction scheme of a LMWC-BR conjugate of the present disclosure.

FIG. 2B is a schematic diagram of a preparation method for the LMWC-BR conjugate of the present disclosure.

FIG. 3 shows the absorbance for each wavelength of the LMWC-BR conjugate of the present disclosure.

FIG. 4 shows a particle diameter distribution of nanoparticles composed of the LMWC-BR conjugate of the present disclosure.

FIG. 5 shows H¹-NMR analysis data of the LMWC-BR conjugate of the present disclosure and low molecular weight chitosan.

FIG. 6 confirms the concentration of the LMWC-BR conjugate of the present disclosure by measuring UV/Vis absorbance.

FIG. 7 shows the particle size by day in an aqueous solvent to identify the stability of nanoparticles composed of the LMWC-BR conjugate of the present disclosure.

FIG. 8 shows the particle size by concentration to identify the concentration of grain boundary micelles of the nanoparticles composed of the LMWC-BR conjugate of the present disclosure.

FIG. 9 compares solubility in each solvent after high molecular weight chitosan (HMWC), low molecular weight chitosan (LMWC), bilirubin (BR), and a low molecular weight chitosan-bilirubin conjugate (LMWC-BR) were dissolved in water and DMSO.

FIGS. 10A, 10B, and 10C show UV/Vis absorbance of the LMWC-BR conjugate of the present disclosure in contact with hydrogen peroxide (H₂O₂) (FIG. 10A), AAPH (FIG. 10B), and NaOCl (FIG. 10C) to identify the antioxidative efficacy of the LMWC-BR conjugate.

FIGS. 11A, 11B, and 11C are graphs for illustrating the ROS-scavenging effect of LMWC-BR conjugate. FIG. 11A is a graph illustrating the fluorescence intensity according to the concentration of H₂O₂, and FIG. 11B shows the concentration of H₂O₂ according to the concentration of LMWC treated with H₂O₂. FIG. 11C shows the concentration of H₂O₂ according to the concentration of the LMWC-BR conjugate treated with H₂O₂.

FIGS. 12A, 12B, 12C and 12D show cell viability when CHO cells and HT-29 cells were treated with LMWC and the LMWC-BR conjugate together with H₂O₂, respectively, in order to identify the ROS scavenging effect of the chitosan-bilirubin conjugate of the present disclosure.

FIG. 13 confirms mRNA expression levels of early-inflammatory cytokines (IL-1beta, IL-6, and TNF-alpha) through RT-qPCR after macrophages were treated with LPS.

FIGS. 14A, 14B, and 14C confirm mRNA expression levels of early-inflammatory cytokines (IL-1beta, IL-6, and TNF-alpha) through RT-qPCR after macrophages were treated with LMWC or LMWC-BR conjugate (2.5 μg/ml, 5 μg/ml, and 10 μg/ml) along with LPS.

FIG. 15 is a schematic diagram of a test method for identifying the mRNA levels of early-inflammatory cytokines (IL-1beta, IL-6, and TNF-alpha) after the macrophages were treated with LMWC or LMWC-BR conjugate, washed, and then treated with LPS.

FIGS. 16A, 16B, and 16C show mRNA levels of early-inflammatory cytokines (IL-1beta, IL-6, and TNF-alpha) after macrophages were treated with LMWC or LMWC-BR conjugate (2.5 μg/ml, 5 μg/ml, and 10 μg/ml), washed, and then treated with LPS.

FIGS. 17A, 17B and 17C show mRNA levels of early-inflammatory cytokines (IL-1beta, IL-6, and TNF-alpha) after macrophages were treated with LMWC-BR conjugate (10 μg/ml) of the present disclosure, washed, and then treated with LPS.

FIG. 18 shows mRNA expression levels of the anti-inflammatory cytokines IL-10 and TGF-beta after macrophages were treated with LMWC-BR conjugate of the present disclosure and LPS.

FIG. 19 is a schematic diagram of the experimental schedule of Example 6-1.

FIG. 20 shows the body weight change for each mouse group in Example 6-1.

FIG. 21 shows the colon length for each mouse group in Example 6-1.

FIG. 22A shows the administration dose for each experimental group in Example 6-2.

FIG. 22B shows the experimental schedule and appearances of substances to be administered in Example 6-2.

FIG. 23 shows the body weight changes by day after the administration of the LMWC-BR conjugate of the present disclosure, LMWC, and BR in a DSS-induced IBD mouse model. The omitted legends of FIG. 23 are the same as those of FIG. 24.

FIG. 24 shows the disease activity index change after the administration of the LMWC-BR conjugate of the present disclosure, LMWC, and BR in a DSS-induced IBD mouse model.

FIG. 25 shows the colon length after the administration of the LMWC-BR conjugate of the present disclosure, LMWC, and BR in a DSS-induced IBD mouse model.

FIG. 26 shows the secretion level of the inflammatory cytokine protein IL-1beta after the administration of the LMWC-BR conjugate of the present disclosure, LMWC, and BR in a DSS-induced IBD mouse model.

FIG. 27 shows the secretion level of the inflammatory cytokine protein IL-6 after the administration of the LMWC-BR conjugate of the present disclosure, LMWC, and BR in a DSS-induced IBD mouse model.

FIG. 28 shows the secretion level of the inflammatory cytokine protein TNF-alpha after the administration of the LMWC-BR conjugate of the present disclosure, LMWC, and BR in a DSS-induced IBD mouse model.

FIG. 29 shows the secretion level of the inflammatory cytokine protein IL-10 after the administration of the LMWC-BR conjugate of the present disclosure, LMWC, and BR in a DSS-induced IBD mouse model.

FIG. 30 shows the secretion level of the inflammatory cytokine protein TGF-beta after the administration of the LMWC-BR conjugate of the present disclosure, LMWC, and BR in a DSS-induced IBD mouse model.

FIG. 31 shows a schematic experimental method of Example 7.

FIG. 32 shows the body weight change for each mouse group in Example 7.

FIG. 33 shows the disease activity index (DAI) for each mouse group in Example 7.

FIG. 34 shows the colon length for each mouse group in Example 7.

FIG. 35 shows the spleen weight for each mouse group in Example 7.

FIG. 36 shows blood ALT and blood AST, which are indicators of liver inflammation scores for each mouse group in Example 7.

FIG. 37 shows blood creatine and blood BUN, which are indicators of whether the renal function was normal, for each mouse group in Example 7.

FIGS. 38 and 39 show the levels of blood IL-6 and blood TNF-alpha, which are indicators of the presence or absence of systemic inflammation, for each mouse group in Example 7.

FIGS. 40, 41 and 42 show the expression levels of ZO-1, Claudin-1, and Occludin-1, which are indicators of intestinal damage in inflammatory bowel disease for each mouse group in Example 7.

FIG. 43 is a schematic diagram of the preparation procedure of a hyaluronic acid-bilirubin conjugate.

FIG. 44 is a diagram showing H1-NMR data of a hyaluronic acid-bilirubin conjugate.

FIG. 45 is a diagram showing the size of nanoparticles through DLS measurement after preparing a hyaluronic acid-bilirubin conjugate as nanoparticles in an aqueous solvent.

FIG. 46 is a diagram showing the measurement of UV absorbance of bilirubin in order to calculate the bilirubin content (weight %) of LMWC-BR and HA-BR.

FIG. 47 is a schematic diagram of the experimental method of Example 8-2 of the present disclosure.

FIG. 48 shows the body weight change for each mouse group in Example 8-2.

FIG. 49 shows the disease activity index (DAI) for each mouse group in Example 8-2.

FIG. 50 shows the colon length for each mouse group in Example 8-2.

FIG. 51 shows the spleen weight for each mouse group in Example 8-2.

FIG. 52 is a schematic diagram of production method for a medium molecular weight chitosan-conjugated bilirubin nanoparticle and a high molecular weight chitosan-conjugated bilirubin nanoparticle.

FIG. 53 shows hydrodynamic size (A) and zeta potential (B) of the synthesized MMWC-BRNPs and HMWC-BRNPs.

FIG. 54 shows amount of bilirubin (BR) in nanoparticle (HMWC-BRNPs, MMWC-BRNPs, LMWC-BRNPs) by measuring UV/vis absorbance at a wavelength of 450 nm.

FIG. 55 shows water solubility according to the molecular weight of chitosan. LMWC and LMWC-BRNPs show higher water solubility.

FIG. 56 shows mucoadhesive properties of each nanoparticle (HMWC-BRNPs, MMWC-BRNPs, LMWC-BRNPs, PEG-BRNPs, and 10 K HA-BRNPs) based on mucin binding efficiency (%).

FIGS. 57A, 57B, 57C, 57D, and 57E show therapeutic effect of chitosan-based BRNPs.

FIG. 58 shows therapeutic efficacy of chitosan-based BRNPs in NASH.

FIG. 59 shows preventive effects on liver and spleen in murine NASH model compared to the MCD diet mice.

FIG. 60 shows anti-inflammatory effect of LMWC-BRNPs in the NASH model.

FIG. 61 shows evaluations for liver damage markers in serum samples.

FIG. 62 shows fat accumulation in serum of LMWC-BRNPs administrated mice.

FIG. 63 shows fat accumulation in liver of LMWC-BRNPs administrated mice.

FIG. 64 shows fat-modulating effect of LMWC-BRNPs.

FIG. 65 shows efficiency of LMWC-BRNP delivery from gut to liver axis.

### DETAILED DESCRIPTION

Hereinafter, the present disclosure will be described in more detail with reference to exemplary embodiments. These exemplary embodiments are provided only for the purpose of illustrating the present disclosure in more detail, and therefore, according to the purpose of the present disclosure, it would be apparent to a person skilled in the art that these exemplary embodiments are not construed to limit the scope of the present disclosure.

### EXAMPLES

Throughout the present specification, the “%” used to express the concentration of a specific material, unless otherwise particularly stated, refers to (wt/wt) % for solid/solid, (wt/vol) % for solid/liquid, and (vol/vol) % for liquid/liquid.

### Example 1: Synthesis of LMWC-BR

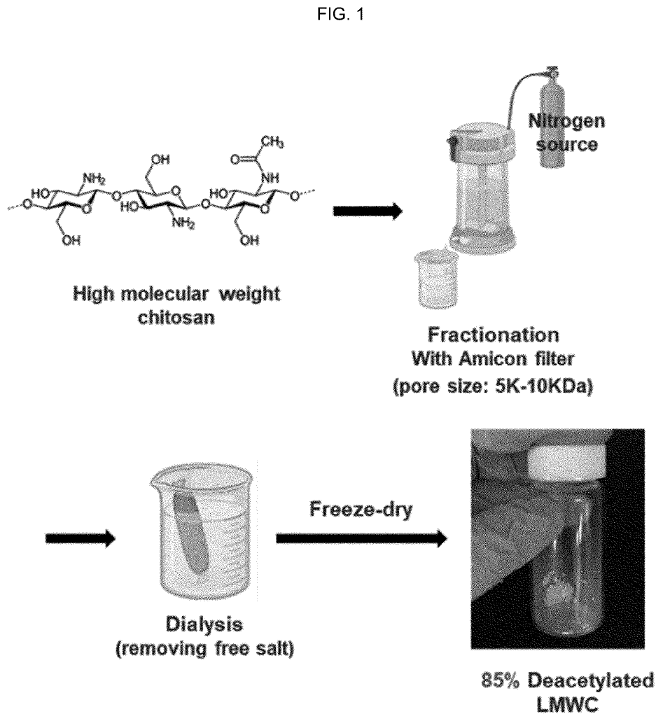

Chito-oligosaccharide (COS) was dissolved in 180 ml of 0.05 ammonium acetate buffer at pH 4.2. The solution was fragmented to small molecules by using an Amikon filter of 5,000-10,000 Da, and subjected to solution extraction by the applied pressure through nitrogen injection, thereby obtaining a chitosan solution sample. Thereafter, dialysis was performed using a 3500-Da filter membrane for complete removal of free salts or the like. The solution completed dialysis was lyophilized to obtain low molecular weight chitosan (LMWC) in a powder form. The lower the molecular weight of chitosan, the higher the water solubility. The degree of deacetylation (DDA, %) was determined based on NMR data (see Journal of Pharmaceutical and Biomedical Analysis 32, 1149-1158 (2003)). A schematic diagram of the low molecular weight chitosan production method is shown in FIG. 1.

Then, a conjugation process with bilirubin was performed. Chitosan was exposed by deacetylation due to fractionation of the amine group thereof. The amine group may react with a carboxylic acid of bilirubin to form a conjugation form.

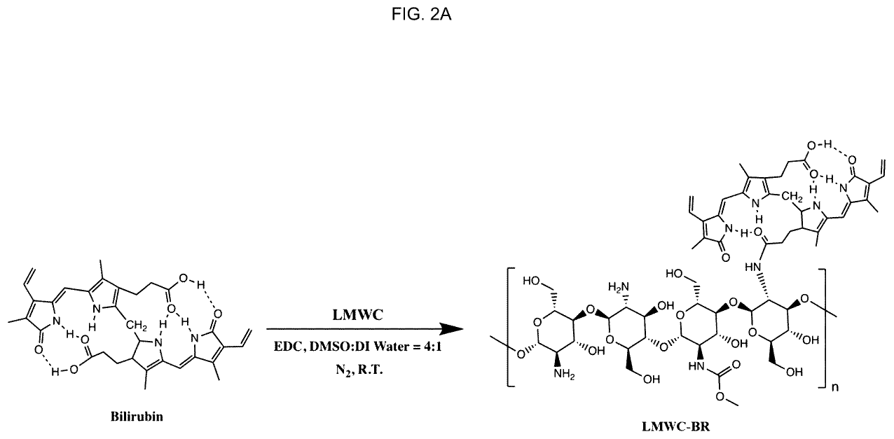

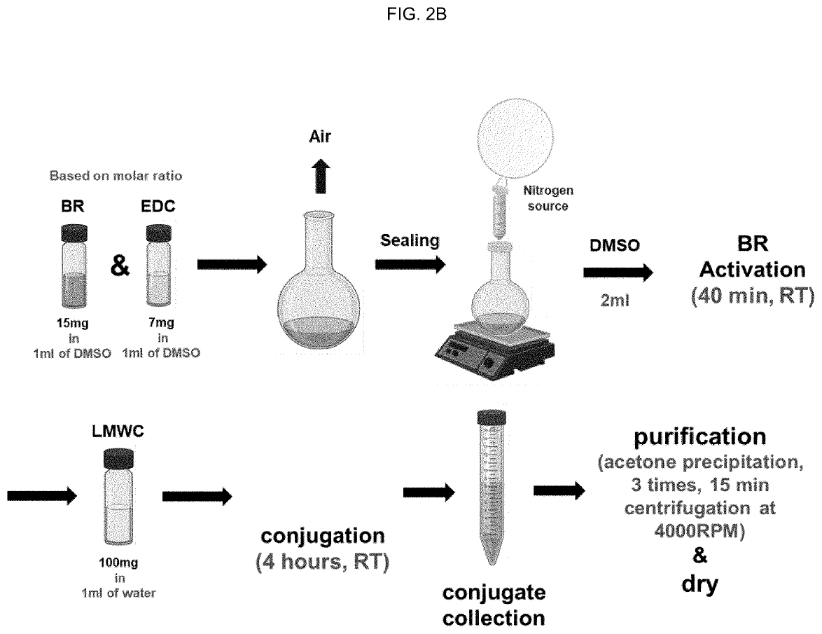

Since the activation of the carboxyl group is required in order for the carboxylic group of bilirubin to react, 1-ethyl-3-(3-dimethylaminopropyl)carbodiimide (EDC) was dissolved with bilirubin in dimethyl sulfoxide (DMSO):water (4:1) to be activated for 40 minutes. Thereafter, the low molecular weight chitosan dissolved in water was added to the reaction, followed by conjugation of chitosan and bilirubin for a total of 4 hours. All reactions were carried out on an agitator while oxygen (air was removed with a vacuum pump and then nitrogen was injected) and light were blocked, and the reaction was carried out in a round flask. Chitosan, bilirubin, and EDC may be mixed at a ratio of 1:1-5:1.5-7.5, and the reaction was carried out by addition at optimal ratio was 1:1:1.5. The reaction scheme of the reaction is shown in FIG. 2A, and a preparing method for a low-molecular-weight chitosan-bilirubin conjugate is schematized in FIG. 2B.

After the conjugation reaction was completed, a process for removing the reactant (EDC) remaining without participating in the reaction was performed. After the reaction, a predetermined amount of the mixture was transferred to a conical tube (15 ml), 10 ml of acetone was added, followed by centrifugation at 4° C. and 4000 rpm for 15 minutes, and the supernatant was discarded. Again, 10 ml of acetone was added, and centrifugation and supernatant discard were repeated two times, and thus a total of three times of centrifugation and supernatant discard were performed. The final product was dried to obtain a brown powdery material and stored in a freezer.

### Example 2: Characterization of LMWC-BR Conjugate

2-1. UV/Vis Spectrum (300 nm-600 nm)

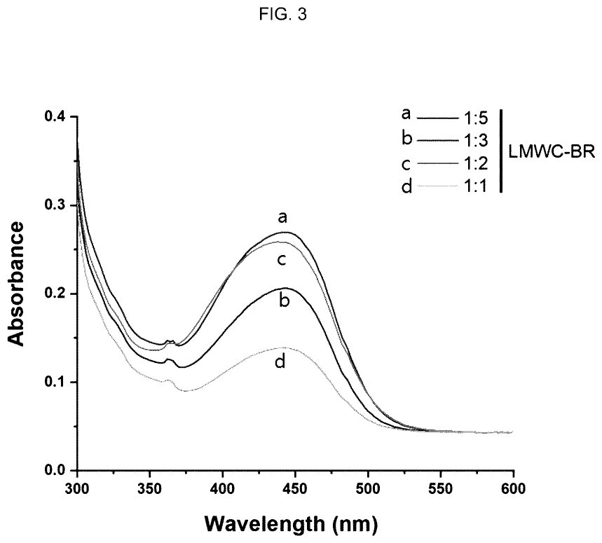

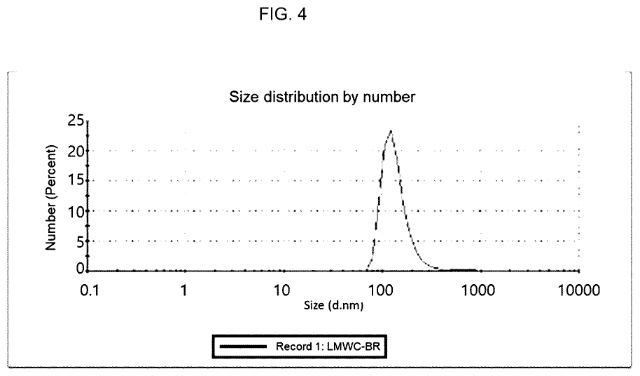

In order to investigate whether the low molecular weight chitosan-bilirubin conjugate of the present disclosure was well formed, chitosan-bilirubin in a powder form was dissolved in distilled water (DW) and measured for UV/Vis absorbance to thereby identify the detection of the characteristic peak of bilirubin. Bilirubin generally exhibits a yellow color and shows a maximum absorbance at a wavelength of 450 nm, and thus the absorbance was measured to investigate whether the peak at the same position was detected in the chitosan-bilirubin conjugate. The results are shown FIG. 3.

As shown in FIG. 3, the peaks of bilirubin were detected at around 430 nm in chitosan-bilirubin conjugate material. The peaks of chitosan were also detected (now shown) at 200 nm to 300 nm, indicating that conjugates were primarily formed.

2-2. Hydrodynamic Size Measurement by DLS

The prepared chitosan-bilirubin conjugate material was dissolved in distilled water, and then in order to measure the size of particles manufactured of the chitosan-bilirubin conjugate material, the diameter of the hydrated particle (hydro-dynamic size) was measured through dynamic light scattering (DLS).

As a result of the measurement, among all the particles present in the solution, the size of particles that occupy the largest proportion was 149.6 nm, and the proportion thereof was highest at 96.2%.

2-3. H1-NMR Spectrum

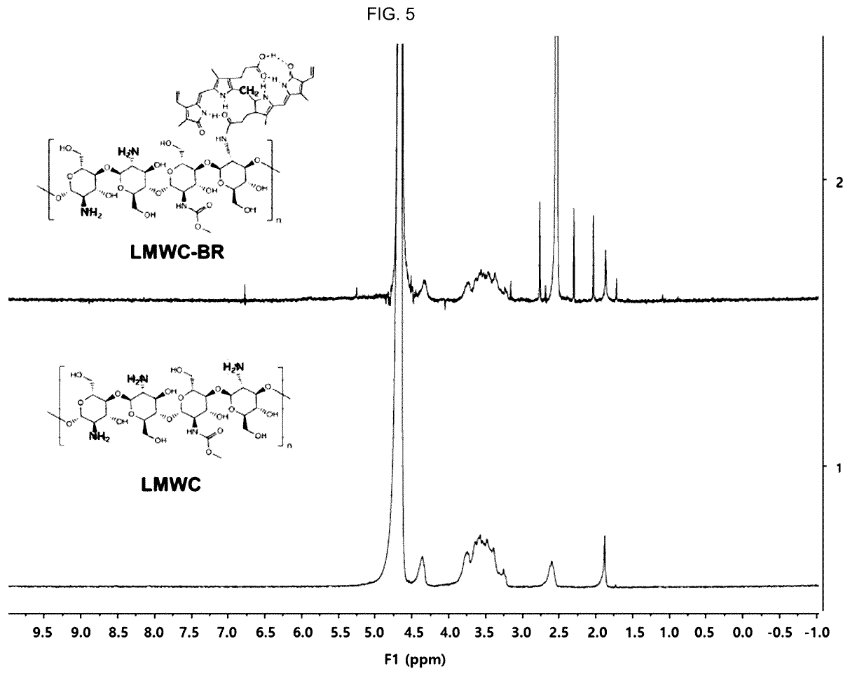

As shown in FIG. 5, the wavelength of chitosan-bilirubin was identified through H1-NMR.

2-4. Calculation of BR Amount in LMWC-BR

TABLE 1

| | | | | Amount of BR |
|---|---|---|---|---|
| | Absorbance | Concentration | Calculated | in 1 mg of |
| | Intensity | of conjugate | BR content | conjugate |
| Sample | (λ = 450 nm) | (mg/ml) | (mg/ml) | (mg) |
| 1:1 | 0.4787 | 0.1 | 0.025 | 0.25 |
| LMWC-BR | | | | |

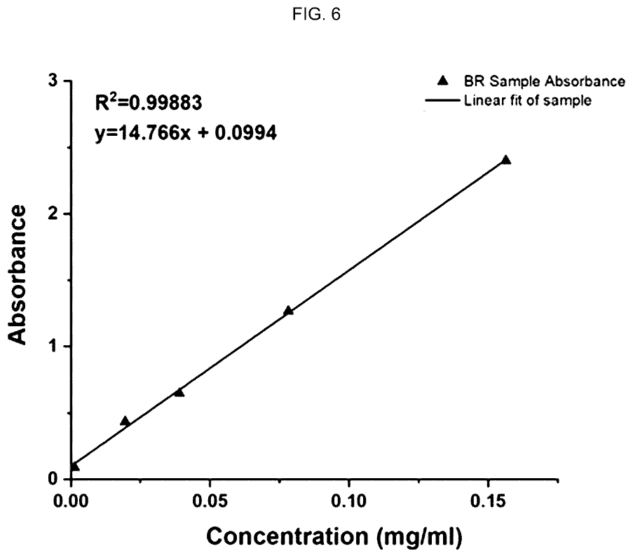

Last, in order to quantify the amount of bilirubin in the chitosan-bilirubin conjugate material of the present disclosure, free-bilirubin was dissolved in DMSO and serially diluted 10-fold to prepare bilirubin solutions of various concentrations, each of which was then measured for UV/Vis absorbance to create a bilirubin purification graph. Through the graph, the concentration of the chitosan-bilirubin conjugate of the present disclosure showing the same absorbance was checked to finally determine the concentration of bilirubin in the chitosan-bilirubin conjugate. The results are shown in FIG. 6 and Table 1.

As shown in FIG. 6 and Table 1, 0.25 mg (weight percentage: 25%) of bilirubin was contained in 1 mg of the chitosan-bilirubin conjugate of the present disclosure. The present inventors checked the amount of bilirubin in the conjugate by newly determining the absorbance based on bilirubin whenever a new chitosan-bilirubin conjugate was synthesized. Since chitosan was fragmented to a size between 5000 Da and 10,000 Da (assuming that 8000 Da fragments were most according to the Boltzmann distribution), the molecular weight of chitosan cannot be accurately calculated. Therefore, all experiments were performed based on the amount of bilirubin in the chitosan-bilirubin conjugate.

2-5. Particle Stability

Hydrodynamic Size Measurement in Distilled Water

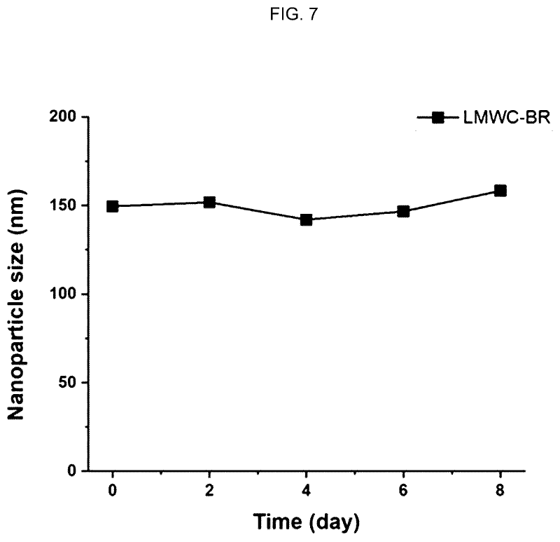

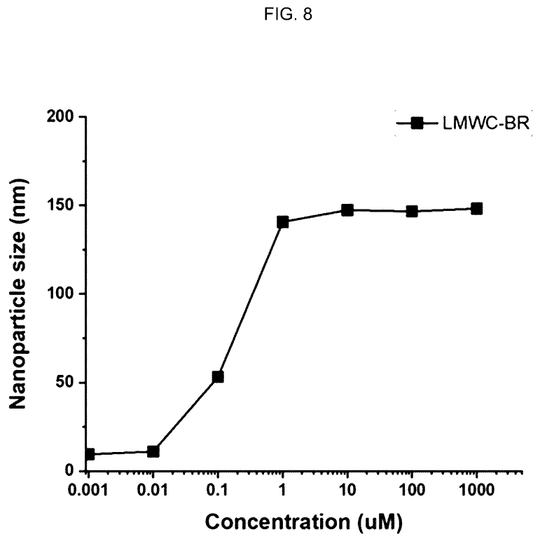

In order to identify the stability, in water, of nanoparticles generated when the chitosan-bilirubin conjugate of the present disclosure was dissolved in water, the chitosan-bilirubin conjugate was dissolved in water, and then the size of the particles was measured using DLS at two-day intervals while refrigerated. In order to find the critical micelle concentration (CMC), the particle size was measured using DLS after dilution at several concentrations by a factor of 10. The results are shown in FIGS. 7 and 8.

As shown in FIG. 7, the nanoparticles manufactured of the chitosan-bilirubin conjugate of the present disclosure in an aqueous solution maintained a constant particle size of about 150 nm for up to 8 days, indicating that highly stable particles were generated.

As shown in FIG. 8, when the concentration of bilirubin was below 1 μM, the size of the particles was decreased or the particles were not well formed.

2-6. Solubility (5 mg in 500 μl of Solvent)

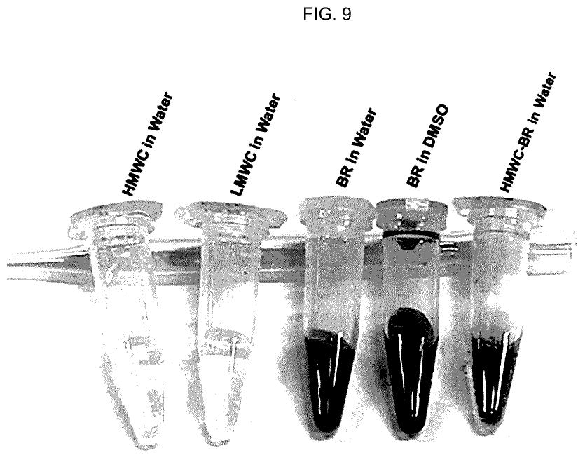

In addition, the present inventors dissolved a high molecular weight chitosan, a low molecular weight chitosan, bilirubin, and a low molecular weight chitosan-bilirubin conjugate in water and DMSO, and then compared the solubility in each solvent. As a result, only the low molecular weight chitosan and the chitosan-bilirubin conjugate were dissolved (dissociation) in water without aggregation (FIG. 9).

### Example 3: ROS-Scavenging Effect of LMWC-BR

3-1. ROS-Scavenging Effect of Low Molecular Weight Chitosan-Bilirubin Conjugate Against Various Kinds of ROS

ROS scavenging effect of BR in conjugate (1000 μM BR in conjugate)

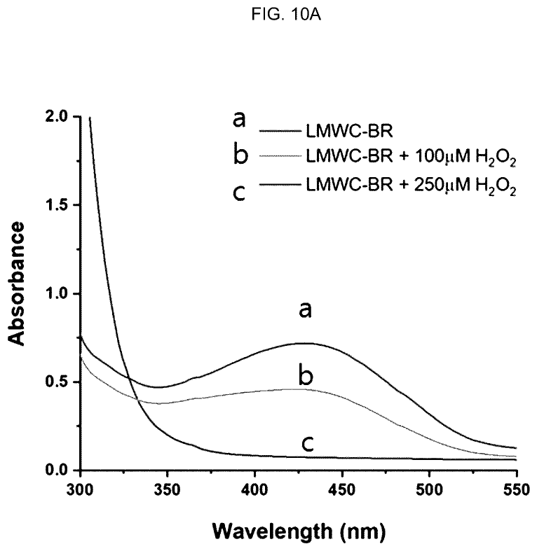

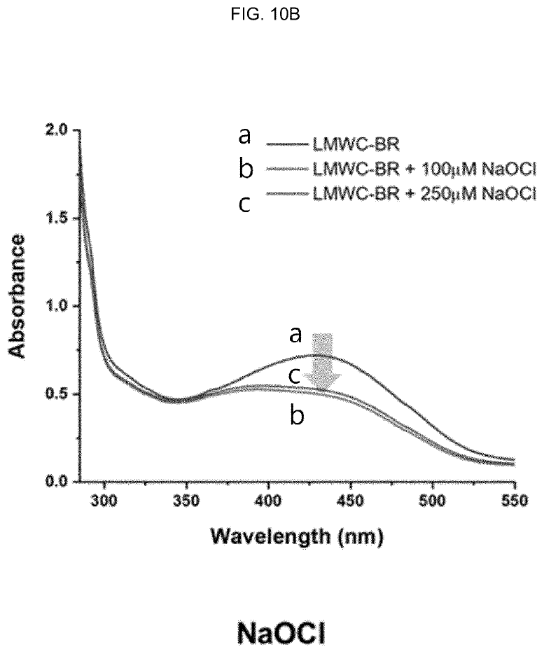

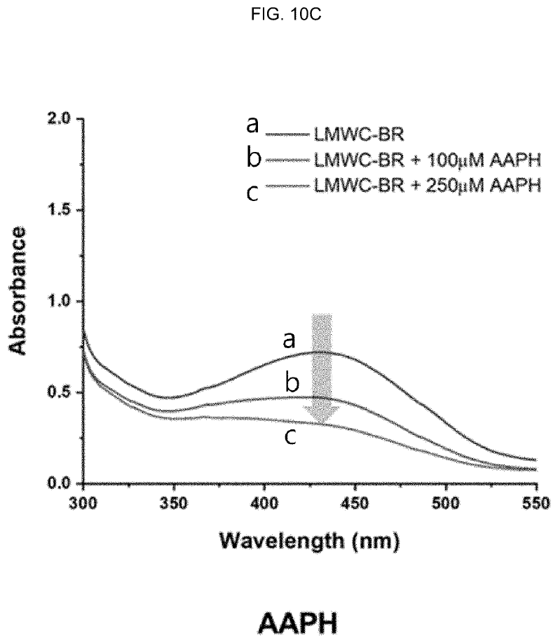

In order to identify whether the low molecular weight chitosan-bilirubin conjugate-containing nanoparticles of the present disclosure could react with ROS, such as reactive oxygen species to reduce ROS, a predetermined amount of low molecular weight chitosan-bilirubin conjugate was reacted with three concentrations (0, 100, and 1000 μM) and three kinds of ROS (hydrogen peroxide, AAPH, and NaOCl) to check the color of the reaction products (if the yellow color disappears or not), and after a predetermined time, the UV/Vis absorbance was measured to check whether the peak of bilirubin disappeared, to verify the reducing ability. The test was performed on the basis of 1000 μM, which was the concentration of bilirubin in the chitosan-bilirubin conjugate-containing nanoparticles. The results are shown in FIGS. 10A to 10C.

As shown in FIGS. 10A to 10C, the peaks of bilirubin were reduced or disappeared in all the three types of ROS, indicating that the low molecular weight chitosan-bilirubin conjugate-containing nanoparticles of the present disclosure had ROS scavenging ability.

3-2. Comparison of ROS-Scavenging Effect Between Low Molecular Weight Chitosan and Inventive Chitosan-Bilirubin Conjugate-Containing Nanoparticles

The present inventors compared the ROS-scavenging effect of the conjugate of the present disclosure with the ROS-scavenging effect of the low molecular weight chitosan itself not conjugated with bilirubin. Specifically, the concentration of H₂O₂ was measured using an HRP assay kit while varying the concentration of H₂O₂. The low molecular weight chitosan (0.2 μM-0.0002 μM) was reacted with 100 μM H₂O₂ for 2 hours, and then the remaining H₂O₂ concentration was detected.

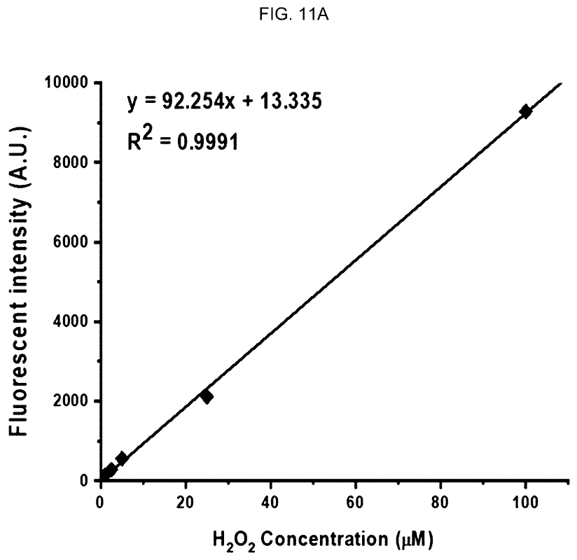

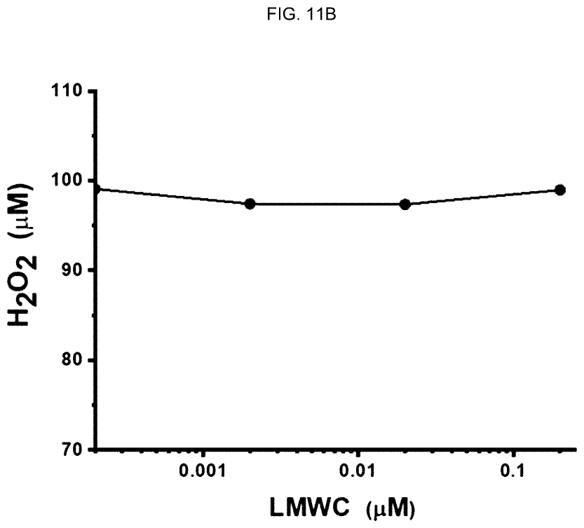

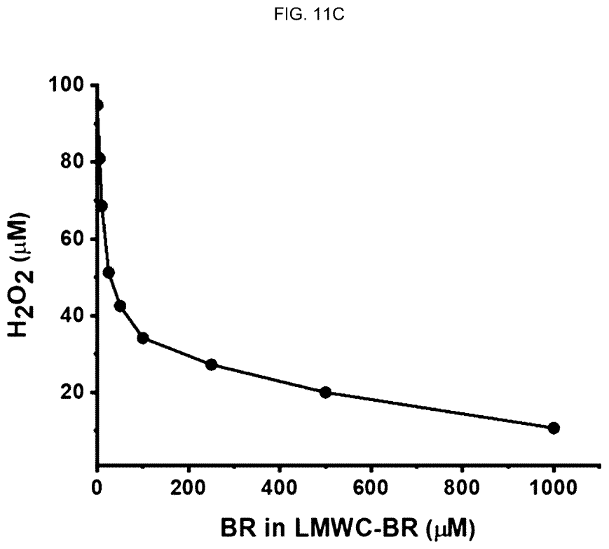

Also, the chitosan-bilirubin conjugate of the present disclosure was added at the same ratio as in the chitosan, and reacted with 100 μM H₂O₂ for 2 hours, and then the remaining H₂O₂ concentration was detected. The results are shown in FIGS. 11A to 11C.

FIGS. 11A to 11C are graphs for illustrating the ROS-scavenging effect of LMWC-BR conjugate. Specifically, FIG. 11A is a graph illustrating the fluorescence intensity according to the concentration of H₂O₂, and FIG. 11B shows the concentration of H₂O₂ according to the concentration of LMWC treated with H₂O₂. FIG. 11C shows the concentration of H₂O₂ according to the concentration of the LMWC-BR conjugate treated with H₂O₂.

As a result of the measurement, the concentration of H₂O₂ was detected to be lower as the concentration of the LMWC-BR conjugate of the present disclosure was higher, and in the group treated with only chitosan, the change in the concentration of H₂O₂ was almost little. Therefore, the LMWC-BR conjugate of the present disclosure had an excellent H₂O₂ scavenging effect, indicating that the H₂O₂ scavenging effect was exhibited by bilirubin conjugated with LMWC.

### Example 4: In Vitro ROS-Scavenging Effect of Low Molecular Weight Chitosan-Bilirubin Conjugate-Containing Nanoparticles (In Vitro Analysis: ROS-Scavenging Effect on Cells)

Cell Cytotoxicity Test

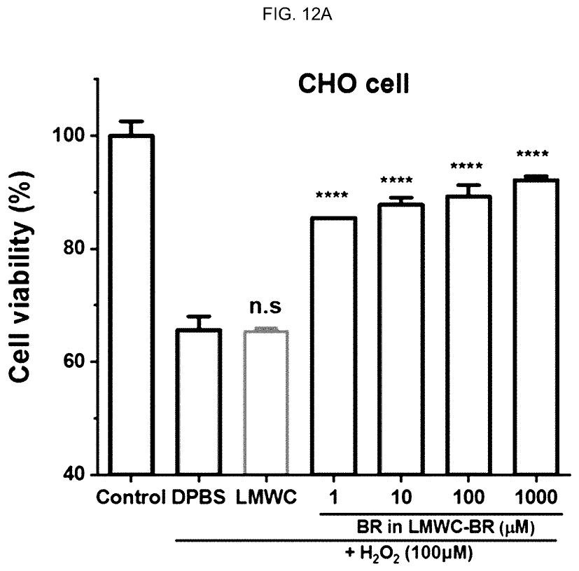

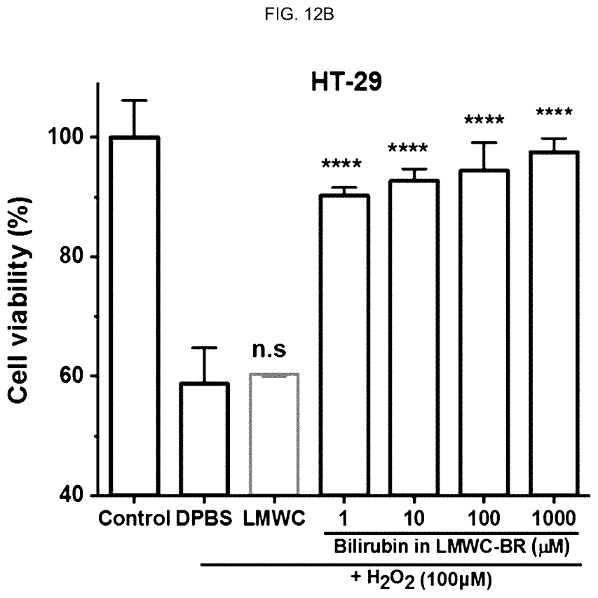

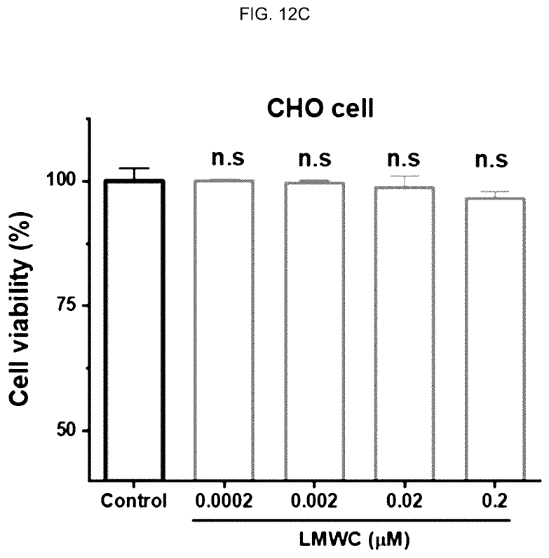

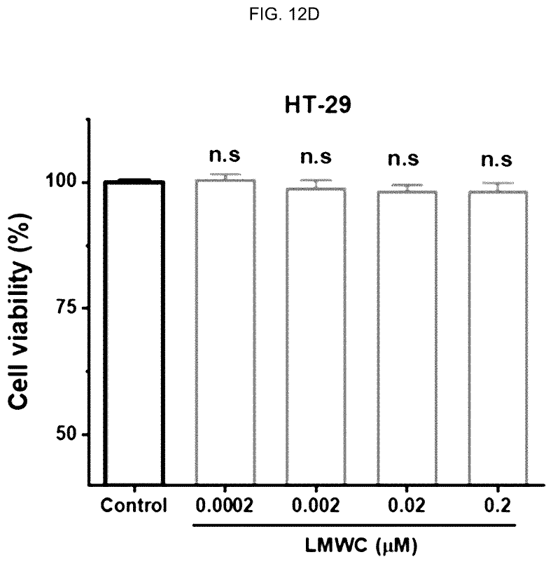

Chitosan-bilirubin conjugate-containing nanoparticles were manufactured at various concentrations (1, 10, 100, and 1000 μM) on the basis of bilirubin, and then Chines Hamster Ovarian cells (CHO cells) and colon cancer cell line HT-29 cells were treated with the nanoparticles, separately. After 5 hours, the cells were treated with 100 μM of H₂O₂, and the change in cell viability due to toxicity by ROS was identified using a WST-8 assay kit. As a comparison group, groups treated with LMWC at various concentrations (0.0002, 0.002, 0.02, and 0.2 μM) instead of LMWC-BR were used. Based on the cell viability of the non-treatment group (control), the cell viability of the other groups was expressed as a percentage. The results are shown in FIGS. 12A to 12D.

As shown in FIGS. 12A to 12D, the group treated with both hydrogen peroxide and chitosan and the group treated only with hydrogen peroxide showed low cell viability, but all the groups treated with the chitosan-bilirubin conjugate of the present disclosure showed high cell viability close to that of the normal group. It can be seen from the above results that the chitosan-bilirubin conjugate of the present disclosure can protect cells by scavenging ROS and has an effect of scavenging H₂O₂ by bilirubin conjugated with LMWC.

### Example 5: Inflammatory Cytokine Expression Inhibitory Effect of Low Molecular Weight Chitosan-Bilirubin Conjugate-Containing Nanoparticles in Macrophages

5-1. Macrophage Activation Pattern in Tissue Injury

In order for the chitosan-bilirubin conjugate of the present disclosure to act as a medicine and exhibit an anti-inflammatory effect, the conjugate needs to have the ability to target key immune cells. The present inventors scheduled future experiments by focusing on macrophages among various immune cells. It is known that macrophages are activated relatively early in the immune response and contribute significantly to immune activation and anti-inflammatory substances involved in the restoration of tissue injury are TGF-beta and IL-10 secreted from macrophages.

5-2. Effects of Chitosan-Bilirubin Conjugate-Containing Nanoparticles of the Present Disclosure on mRNA Expression Levels of Inflammatory Cytokines in Macrophages

Comparison of mRNA Expression Levels of Inflammatory Cytokines in Macrophages after Treatment with LMWC and LMWC-BR

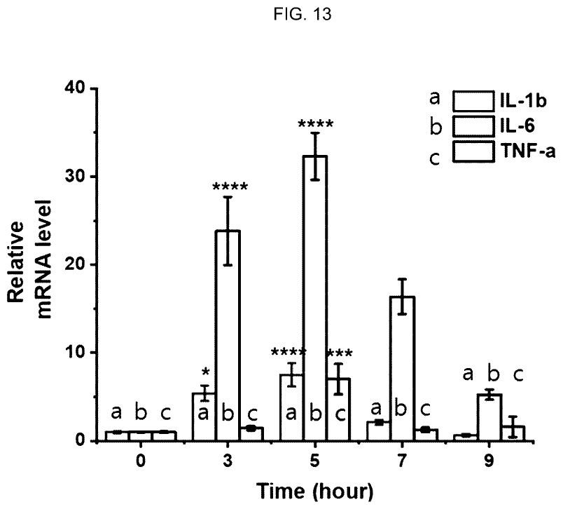

In order to identify the effect of the chitosan-bilirubin conjugate-containing nanoparticles of the present disclosure on the polarization of macrophages, the following test was conducted. First, J774.1 cell line, a type of macrophage, was treated with 0.5 μg/ml of lipopolysaccharide (LPS) to induce inflammation, and then after 0, 3, 5, 7, and 9 hours after LPS treatment, RNA was purified from each cell, and the mRNA expression levels of early-inflammatory cytokines (IL-1beta, IL-6, and TNF-alpha) were determined by RT-qPCR. The results are shown FIG. 13.

As shown in FIG. 13, the mRNA expression levels were measured to be highest 5 hours after treatment of macrophages with LPS. Since mRNA is finally translated into a protein during the expression process, the expression level thereof decreased again after a predetermined time.

Thereafter, for a new experiment, cells were first treated with chitosan (LMWC: 1.875, 3.75, and 7.5 μg/ml) and the chitosan-bilirubin conjugate (LMWC-BR: 0.2, 5, and 10 μg/ml), and after 2 hours, the cells were treated with LPS and incubated for 5 hours, followed by RNA extraction from the cells, and then in the same manner, the expression levels of inflammatory cytokines were compared through RT-qPCR. In the example, the difference between the concentration of chitosan and the concentration of chitosan-bilirubin conjugate was set so that the same amount of chitosan was included in consideration of the content ratio of chitosan/bilirubin according to Table 1, and the same was applied to the following experiments.

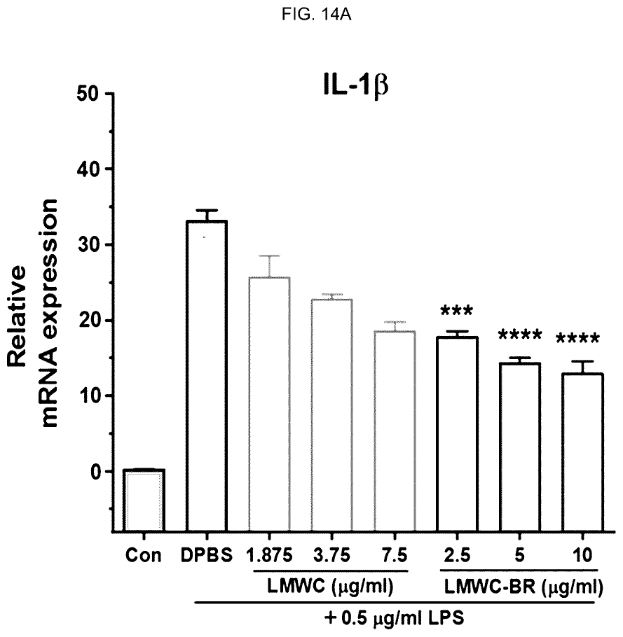

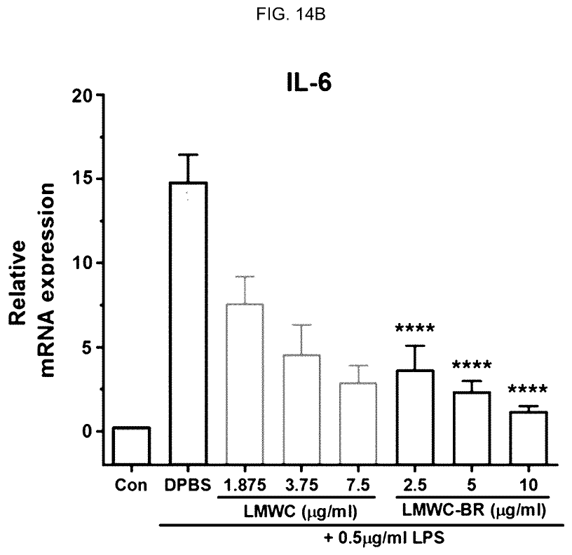

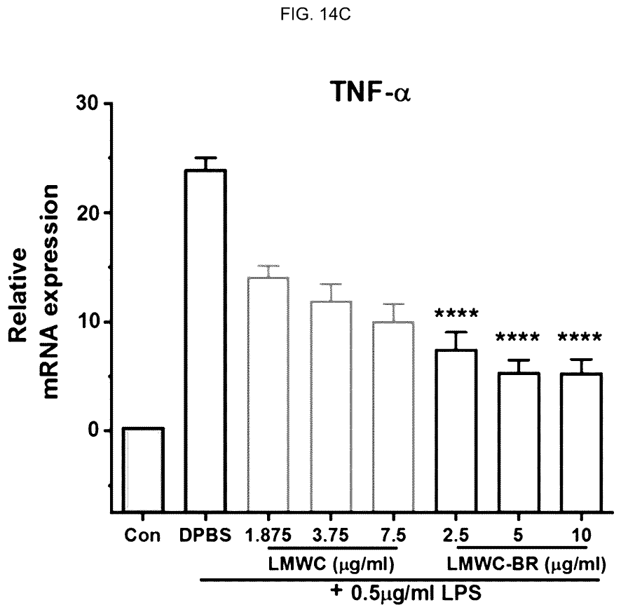

FIGS. 14A to 14C show mRNA expression levels of early-inflammatory cytokines (IL-1beta, IL-6, and TNF-alpha) through RT-qPCR after the cells were treated with LMWC or a LMWC-BR conjugate with LPS.

As shown in FIGS. 14A to 14C, LMWC also exhibited an anti-inflammatory effect in a concentration-dependent manner, and that LMWC-BR of the present disclosure also exhibited an anti-inflammatory effect in a concentration-dependent manner.

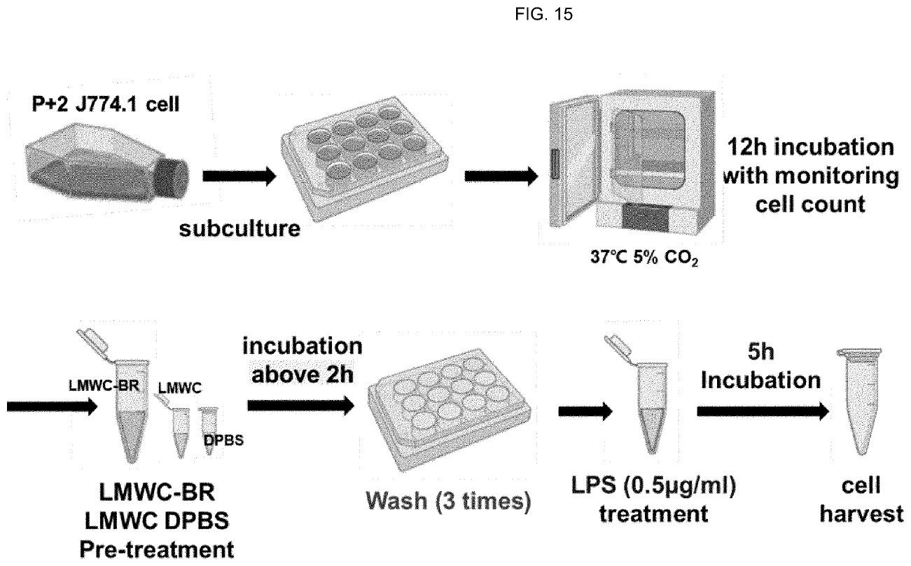

However, chitosan was known to antagonize the inflammation inducing effect of LPS by charge-charge interaction with LDT (Biomaterials 29 (2008) 2173-2182). Specifically, chitosan, like LPS, enters macrophages through the TLR4 receptor to increase inflammatory cytokines, wherein co-treatment with LPS and chitosan allows the reaction of LPS and chitosan, and thus an inflammatory effect disappears. Therefore, in order to obtain more accurate experimental results, the cells were treated with chitosan (LMWC) and the chitosan-bilirubin conjugate (LMWC-BR), as test substances, 2 hours earlier, to prevent chitosan and LPS from co-existing in the cell culture, followed by culturing, and then the cells were thoroughly washed and then treated with LPS (FIG. 15).

Comparison of mRNA Expression Levels of Inflammatory Cytokines in Macrophages after Treatment with LMWC and LMWC-BR (Re-Test Results)

FIG. 15 is a schematic diagram of a test method for identifying the mRNA levels of early-inflammatory cytokines (IL-1beta, IL-6, and TNF-alpha) after the macrophages were treated with LMWC or LMWC-BR conjugate, washed, and then treated with LPS.

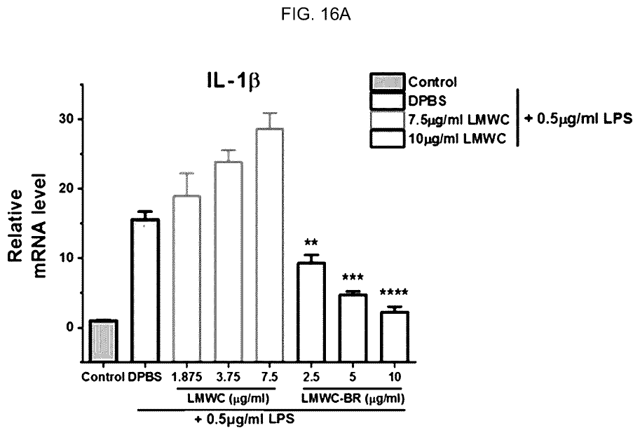

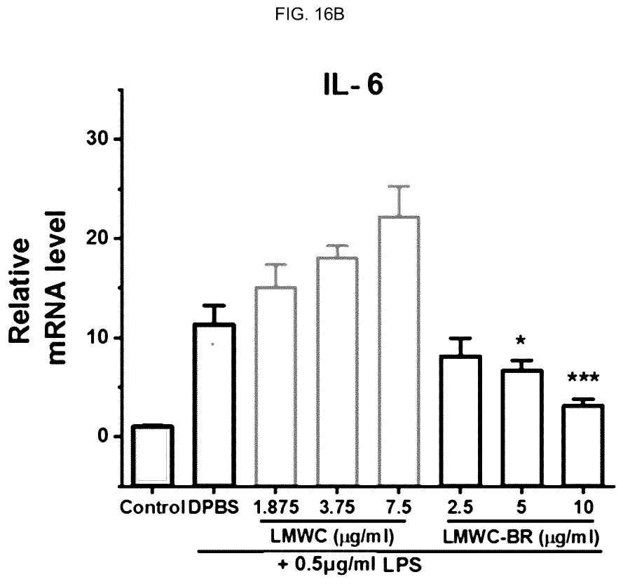

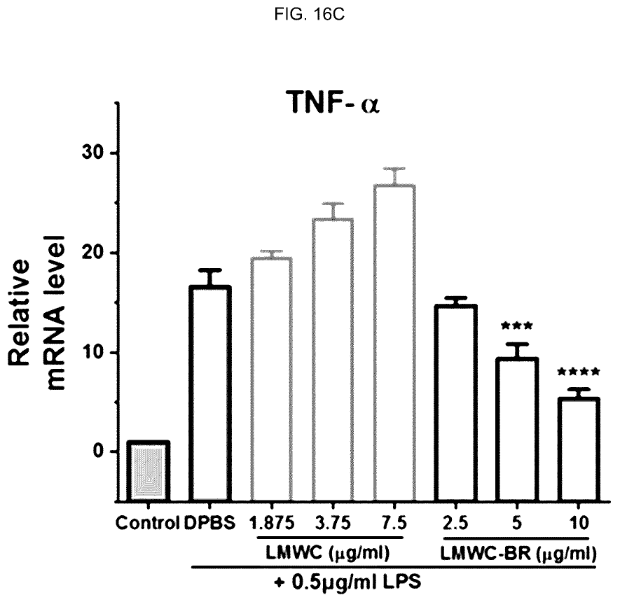

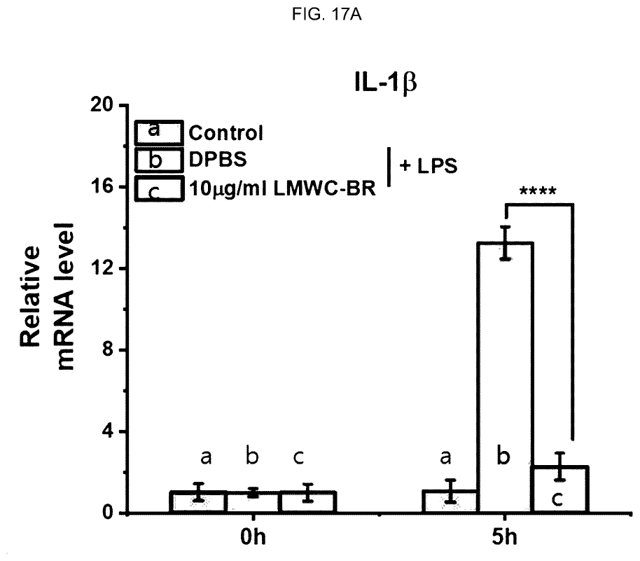

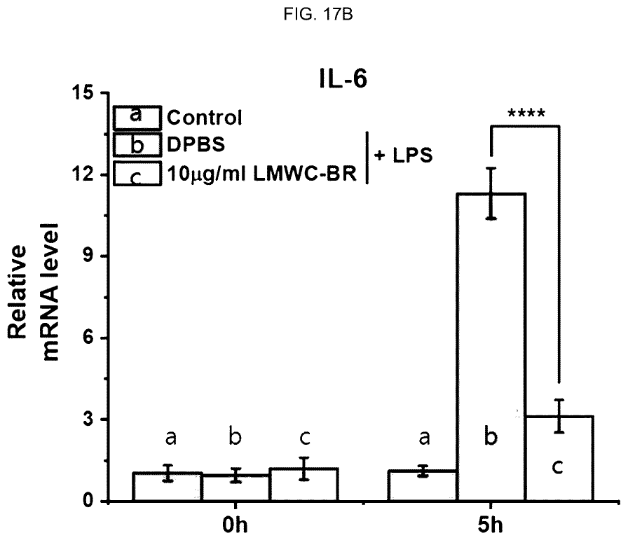

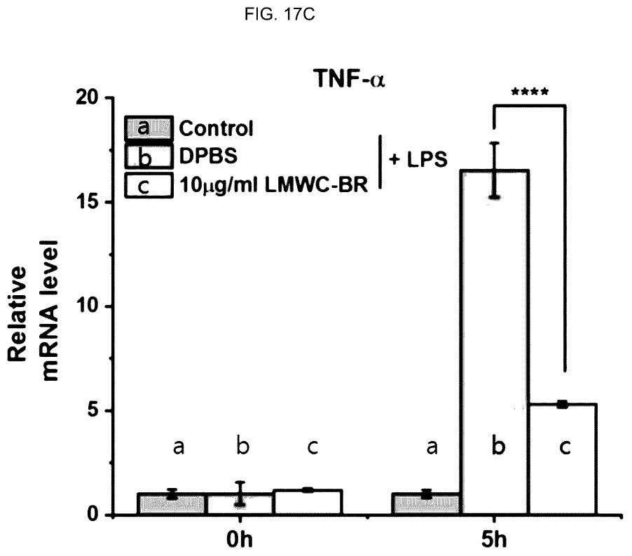

As shown in FIG. 15, the results of the re-experiment according to the order of treatment of changed materials are shown in FIGS. 16A to 16C and FIGS. 17A to 17C.

FIG. 16A to 16C show the mRNA levels of early-inflammatory cytokines (IL-1beta, IL-6, and TNF-alpha) after the macrophages were treated with LMWC or LMWC-BR conjugate, washed, and then treated with LPS.

As shown in FIG. 16A, in the groups treated with only chitosan, IL-1beta increased as the concentration of chitosan increased after 5 hours of LPS treatment, whereas the chitosan-bilirubin conjugate of the present disclosure exhibited an anti-inflammatory effect by reducing the mRNA expression level of IL-1beta in a concentration-dependent manner. As shown in FIGS. 16B and 16C, the expression levels of the inflammatory cytokines IL-6 and TNF-alpha increased as the concentration of chitosan increased after 5 hours of LPS treatment, whereas the chitosan-bilirubin conjugate-containing nanoparticles of the present disclosure reduced the mRNA expression levels of the inflammatory cytokines IL-6 and TNF-alpha in a concentration-dependent manner.

Meanwhile, the macrophages were treated with LMWC-BR of the present disclosure at 10 μg/ml, which was a concentration corresponding to 7.5 μg/ml LMWC, and then the mRNA expression levels of inflammatory cytokines over time in the macrophages (FIGS. 17A to 17C). As a result, the group treated with LMWC-BR of the present disclosure was excellent in the mRNA expression reducing effect of the inflammatory cytokines IL-1beta, IL-6, and TNF-alpha after 5 hours.

5-3. Effects of Chitosan-Bilirubin Conjugate-Containing Nanoparticles of the Present Disclosure on mRNA Expression Levels of Anti-Inflammatory Cytokines in Macrophages (Macrophage Polarization Test)

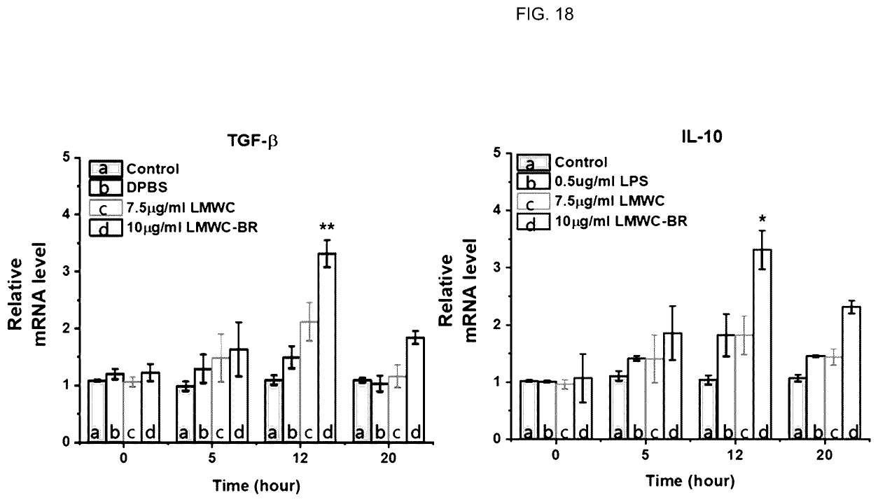

In order to identify the effect of treatment with the chitosan-bilirubin conjugate of the present disclosure on the expression levels of IL-10 and TGF-beta, which are known to result in an anti-inflammatory effect in macrophages, the expression levels of the anti-inflammatory cytokines WL-10 and TGF-beta) over time were examined in the 10 μg/ml LMWC-BR conjugate treatment group showing the best anti-inflammatory effect in the above example and the 7.5 μg/ml (chitosan content corresponding thereto) LMWC treatment group. The results are shown FIG. 18.

As shown in FIG. 18, both the anti-inflammatory cytokines showed the highest expression levels at 12 hours, and the mRNA expression levels of the anti-inflammatory cytokines increased in all the groups treated with LPS. However, the group treated with the chitosan-bilirubin conjugate of the present disclosure, which contained bilirubin, showed the highest increase.

### Example 6: Evaluation on Efficacy of Low Molecular Weight Chitosan-Bilirubin Conjugate-Containing Nanoparticles in Mouse Inflammatory Bowel Disease Model

6-1. In Vivo Efficacy of LMWC-BR in DSS-Induced IBD Mouse Model

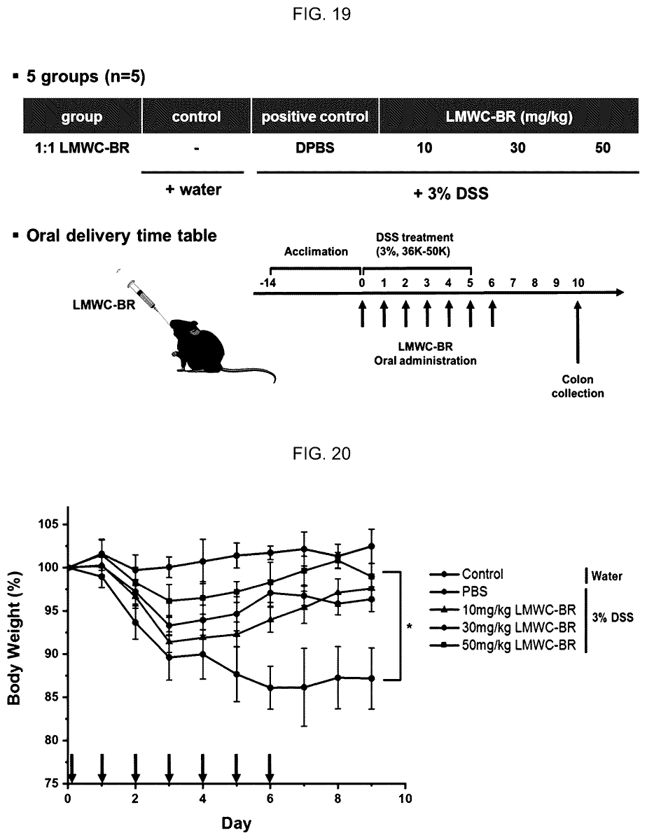

In order to identify the efficacy of the chitosan-bilirubin conjugate of the present disclosure in an IBD mouse model, the following experiment was conducted. First, in order to induce inflammatory bowel disease (IBD) symptoms in mice (C57BL/6, 6 weeks old, female, undergoing a 14-day acclimatization), dextran sulfate sodium (DSS, disrupting the intestinal wall to increase bacterial intake and promote bleeding), which is a substance that causes intestinal inflammation, and a chitosan-bilirubin conjugate, which is a test substance, were orally administered every day for a total of 7 days from the start of administration of DSS. Specifically, the test substance was administered under three administration conditions (10 mg/kg, 30 mg/kg, and 50 mg/kg) to identify the effective dose. The degree of the disease progression and the effect of the chitosan-bilirubin conjugate of the present disclosure were identified by checking the weight change of all mouse groups. The number of times of oral administrations was arbitrarily set for efficacy verification. On the 9th day of the experiment, all the mice were sacrificed to collect the intestine, and in order to identify the degree of inflammation and the efficacy of the chitosan-bilirubin conjugate of the present disclosure, the length from the lower part of the cecum to the rectum was compared. The administration of DSS is characterized by shortening of the intestine due to the occurrence of intestinal inflammation. A schematic experimental schedule of Example 6-1 is shown in FIG. 19.

FIG. 20 shows the body weight change for each mouse group in Example 6-1.

FIG. 21 shows the colon length for each mouse group in Example 6-1.

As shown in FIG. 20, the body weight change was smallest in the group administered the 50 mg/kg chitosan-bilirubin conjugate of the present disclosure.

As shown in FIG. 21, the colon length was longest in the group administered the 50 mg/kg chitosan-bilirubin conjugate of the present disclosure, wherein the colon length was close to that of the normal group.

6-2. Comparison of In Vivo Efficacy of LMWC-BR, LMWC, and BR in DSS-Induced IBD Mouse Model

Body Weight, Disease Activity Index, and Colon Length

The experimental results of Example 6-1 above showed a medicinal effect was the highest at a dose of 50 mg/kg. The present inventors identified whether the effect of the chitosan-bilirubin conjugate of the present disclosure was excellent than the other groups by comparing in vivo efficacy of the 50 mg/kg chitosan-bilirubin treatment group, 12.5 mg/kg free-bilirubin treatment group, and 37.5 mg/kg free-chitosan treatment group on the basis of the results of Example 5-1.

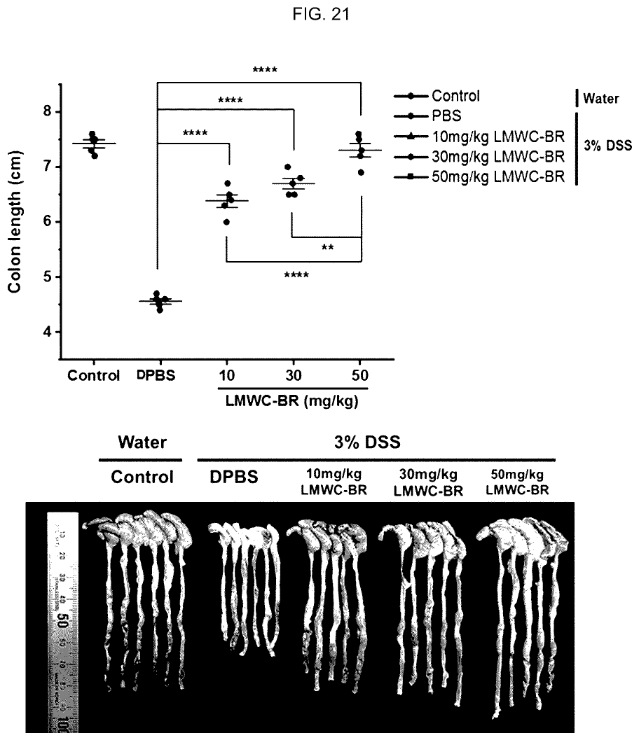

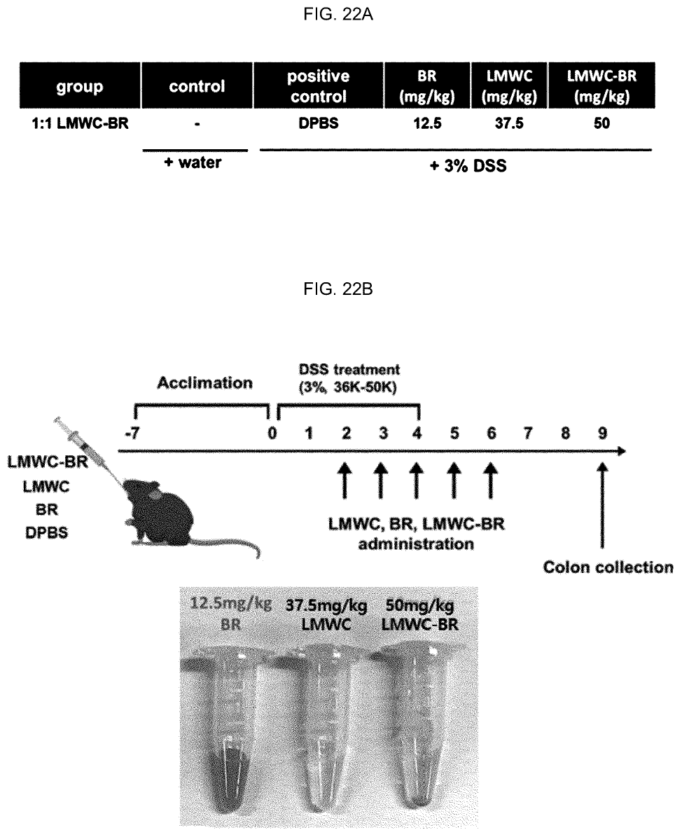

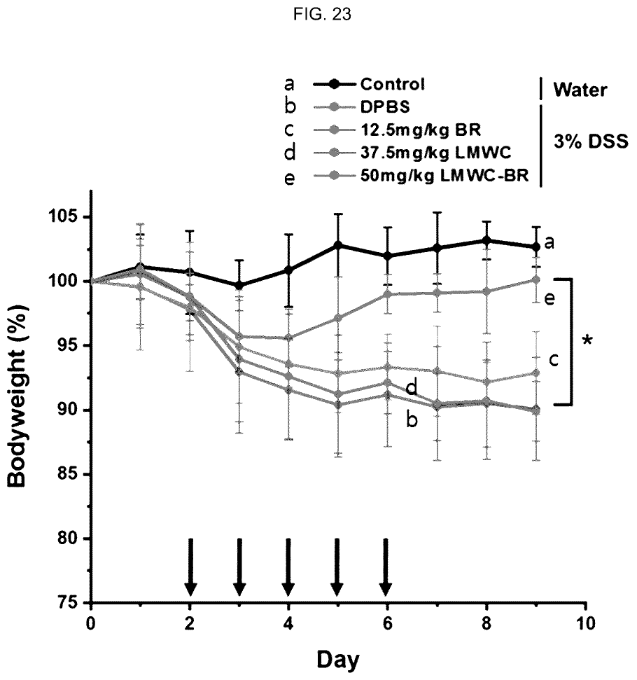

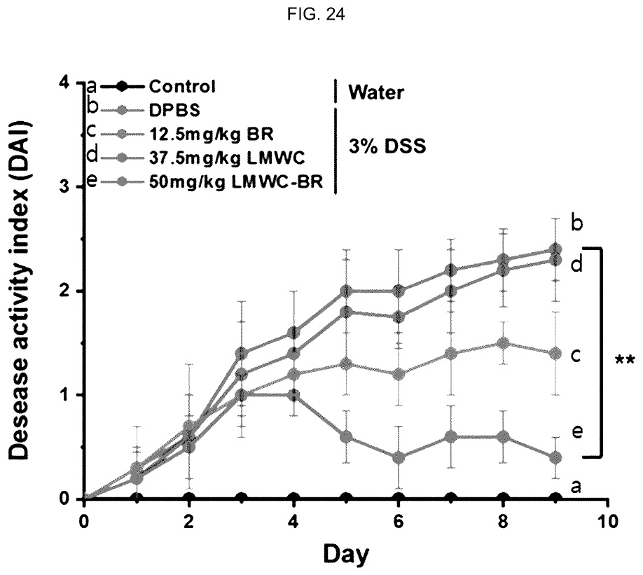

In this Example 6-2, DSS induced enteritis in mice, and then oral administration was performed a total of five times (one administration per day) from the second day when enteritis symptoms began to appear. Bilirubin was not well dissolve din water, but administered in the form of a suspension. Chitosan had a low molecular weight and thus can be injected in the form of being completely dissolved in water (FIG. 22B). The dose of each drug in each group was administered by calculating the ratio of each contained in water (FIG. 22a). The daily weight change was checked for 9 days starting from the day of DSS feeding (FIG. 23), and separately, a step of comparing the disease activity index (Disease Activity Index) by checking the weight loss rate or stool condition was additionally performed (FIG. 24). The criteria for assigning scores were in accordance with Table 2 below. In addition, as in Example 6-1, mice were sacrificed on the 9th day to collect the intestines, and efficacy was evaluated by comparing the length from the lower cecum to the rectum in the same manner.

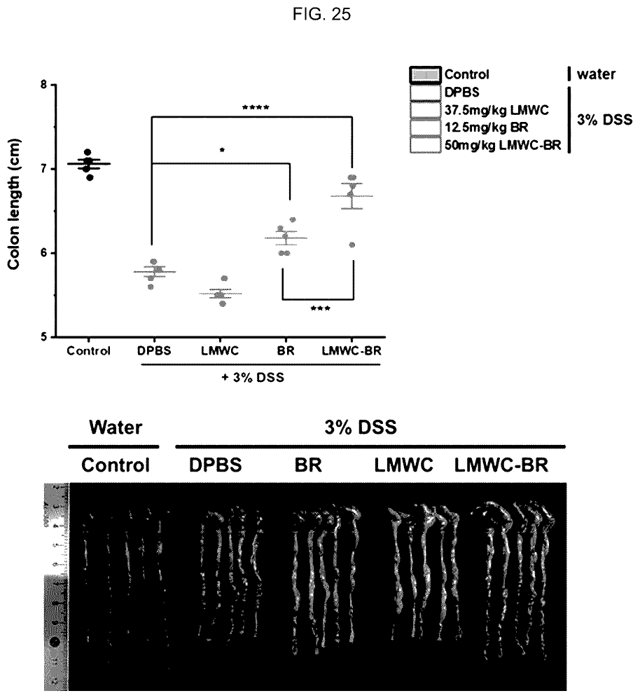

Schematic experimental method in Example 6-2 is shown in FIG. 22A and FIG. 22B. FIG. 23 shows the body weight change for each mouse group in Example 6-2. FIG. 24 shows the disease activity index (DAI) for each mouse group in Example 6-2. FIG. 25 shows the colon length for each mouse group in Example 6-2.

As shown in FIG. 23, the body weight change was smallest in the group administered the 50 mg/kg chitosan-bilirubin conjugate of the present disclosure.

As shown in FIG. 24, the disease activity index was low in the group administered the 50 mg/kg chitosan-bilirubin conjugate of the present disclosure.

As shown in FIG. 25, the colon length was longest in the group administered the 50 mg/kg chitosan-bilirubin conjugate of the present disclosure, wherein the colon length was close to that of the normal group.

TABLE 2

| Score | Body weight loss (%) | Stool consistency | Gross bleeding |
|---|---|---|---|
| 0 | none | normal | normal |
| 1 | 1-5 | — | |
| 2 | 5-10 | soft | hemoccult positive |
| 3 | 10-15 | — | |
| 4 | >15 | watery | Gross bleeding |

Measurement of Intestinal Inflammatory Cytokines (IL-1Beta, IL-6, TNF-Alpha)

Like in the cell experiment of Example 5, the levels of intestinal inflammatory cytokines (IL-1beta, IL-6, TNF-alpha) were determined by ELISA assay.

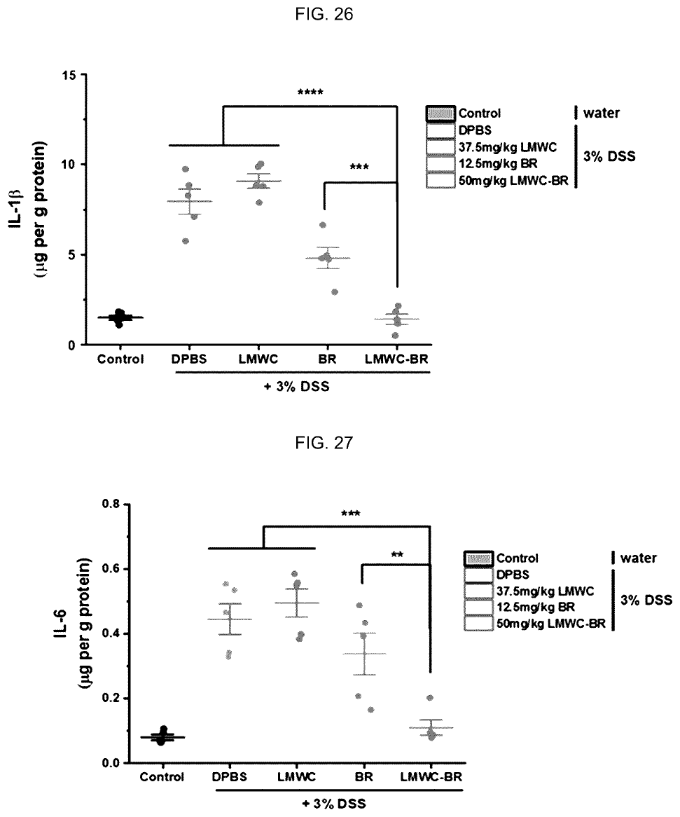

An assay for measuring the protein levels of IL-1beta, IL-6, and TNF-alpha was performed using a supernatant, obtained by homogenizing the same part of the intestine collected for each group of the sacrificed mice through pre-treatment, followed by centrifugation. The results are shown in FIGS. 26 to 28.

As shown in FIGS. 26 to 28, the protein expression levels of inflammatory cytokines were lowest in the 50 mg/kg chitosan-bilirubin treatment group.

Measurement of Intestinal Anti-Inflammatory Cytokines (IL-10 and TGF-β)

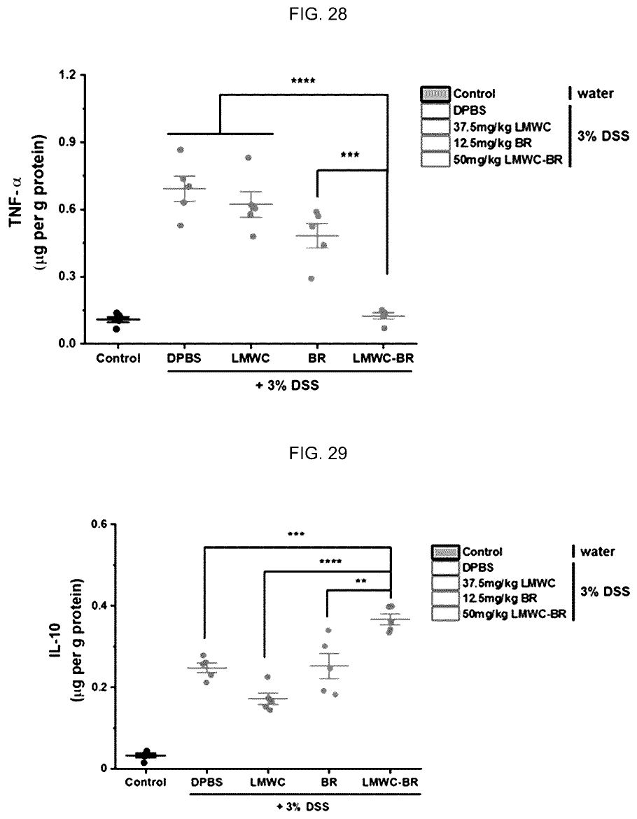

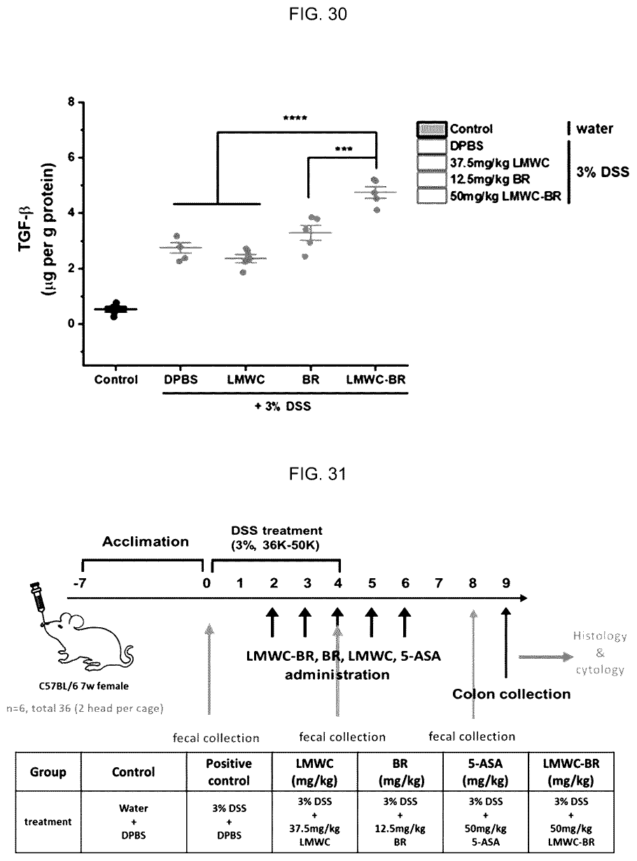

The expression levels of anti-inflammatory cytokines (IL-10 and TGF-β) were also determined in the same manner. The results are shown in FIGS. 29 and 30.

As shown in FIGS. 29 and 30, the protein expression levels of inflammatory cytokines were lowest in the 50 mg/kg chitosan-bilirubin treatment group.

### Example 7: Evaluation on Efficacy of Low Molecular Weight Chitosan-Bilirubin Conjugate-Containing Nanoparticles in Mouse Inflammatory Bowel Disease Model (2)

The present inventors again identified the effect of the chitosan-bilirubin conjugate of the present disclosure by comparing in vivo efficacy of the 50 mg/kg chitosan-bilirubin treatment group, 12.5 mg/kg free-bilirubin treatment group, 37.5 mg/kg free-chitosan treatment group, and the group treated with 5-ASA at an equivalent amount (50 mg/kg) of chitosan-bilirubin as a commercialized drug control group, on the basis of the experimental results of Examples 6-1 and 6-2.

A schematic experimental method of Example 7 is shown in FIG. 31.

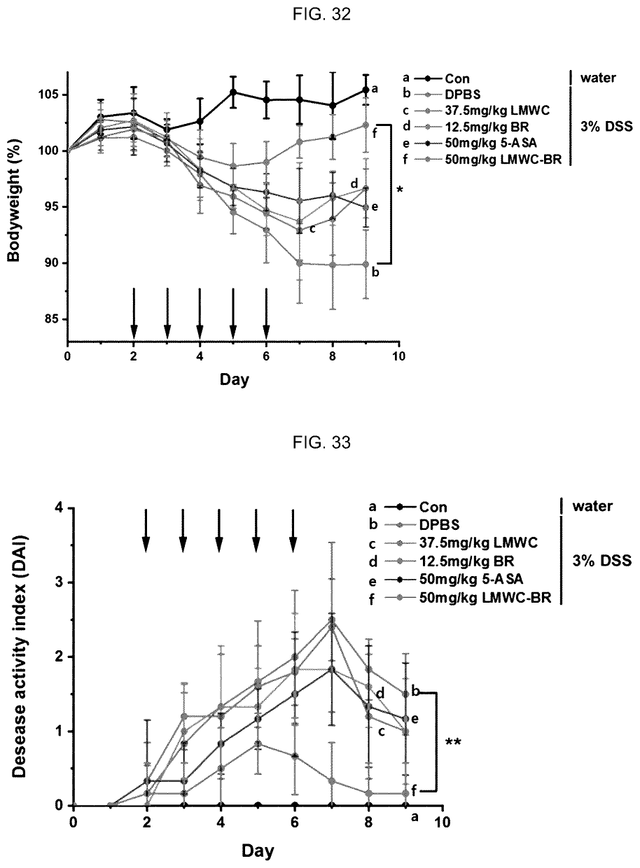

FIG. 32 shows the body weight change for each mouse group in Example 7.

FIG. 33 shows the disease activity index (DAI) for each mouse group in Example 7.

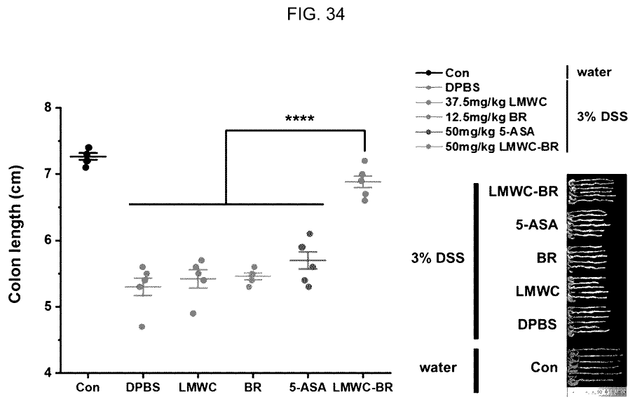

FIG. 34 shows the colon length for each mouse group in Example 7.

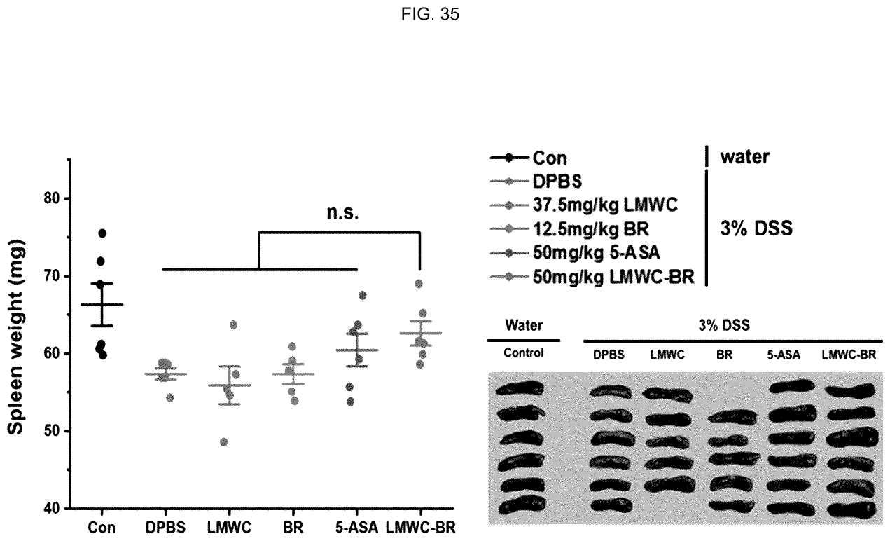

FIG. 35 shows the spleen weight for each mouse group in Example 7.

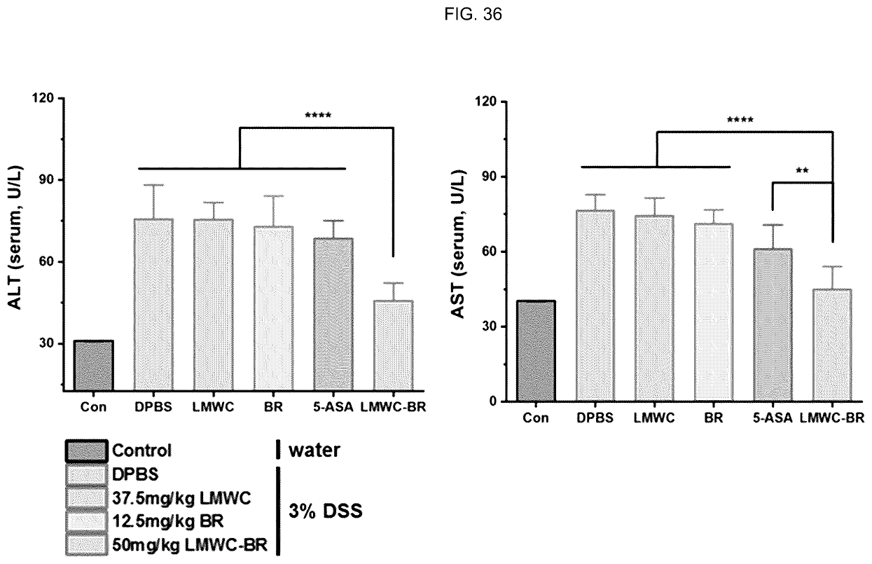

FIG. 36 shows blood ALT and blood AST, which are indicators of liver inflammation scores for each mouse group in Example 7.

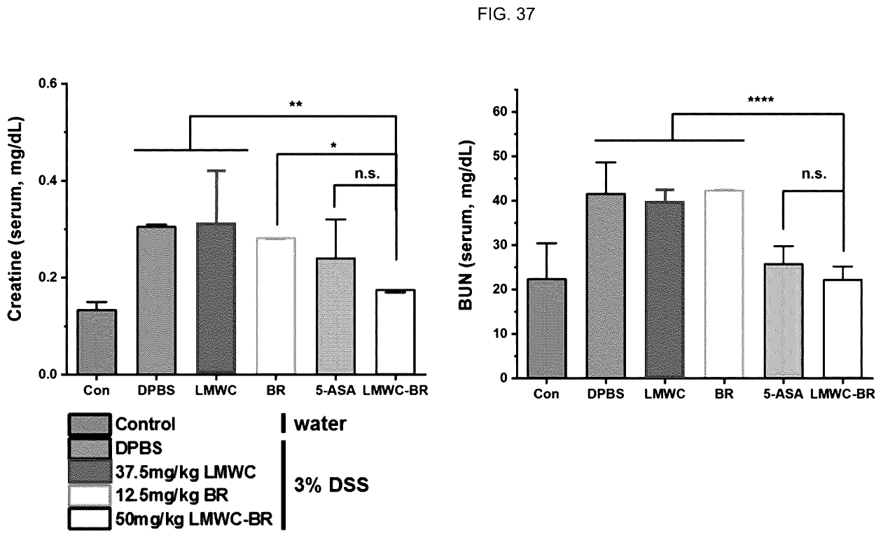

FIG. 37 shows blood creatine and blood BUN, which are indicators of whether the renal function was normal, for each mouse group in Example 7.

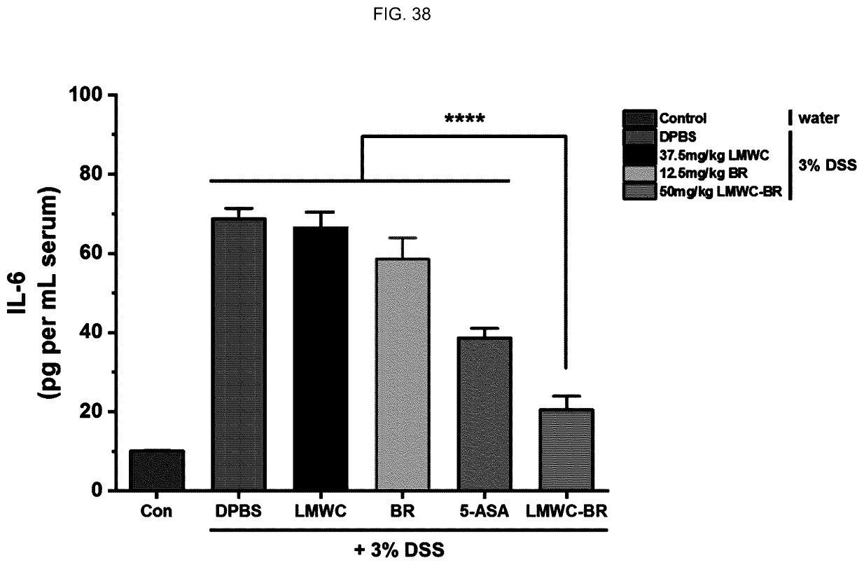

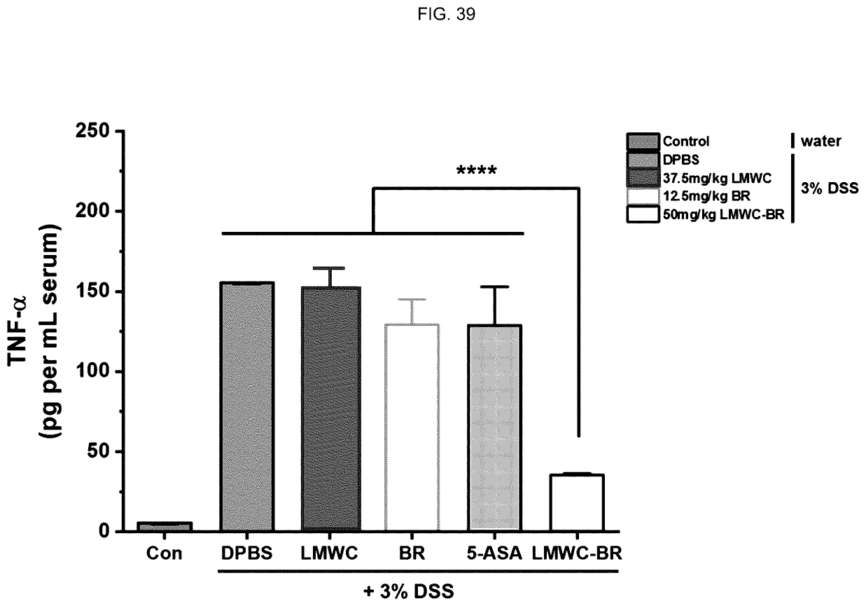

FIGS. 38 and 39 show the levels of blood IL-6 and blood TNF-alpha, which are indicators of the presence or absence of systemic inflammation, for each mouse group in Example 7.

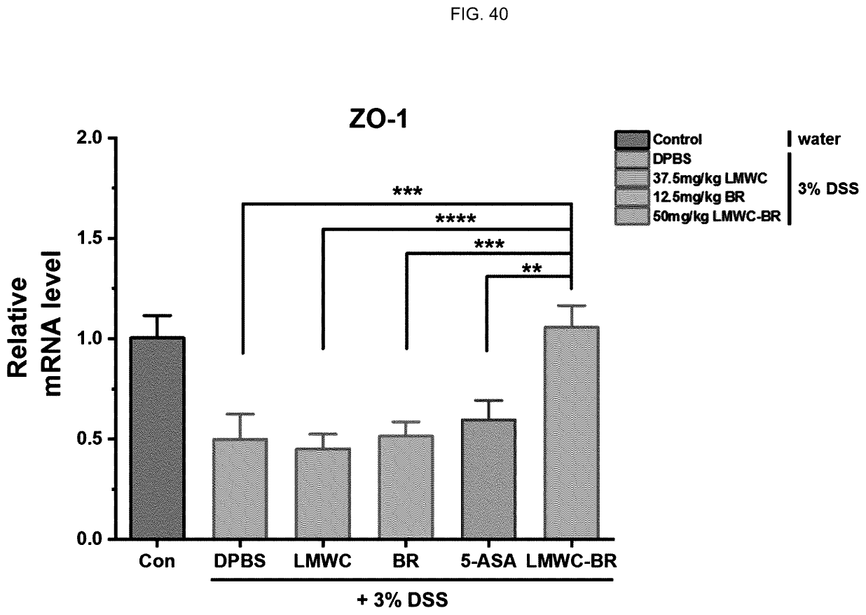

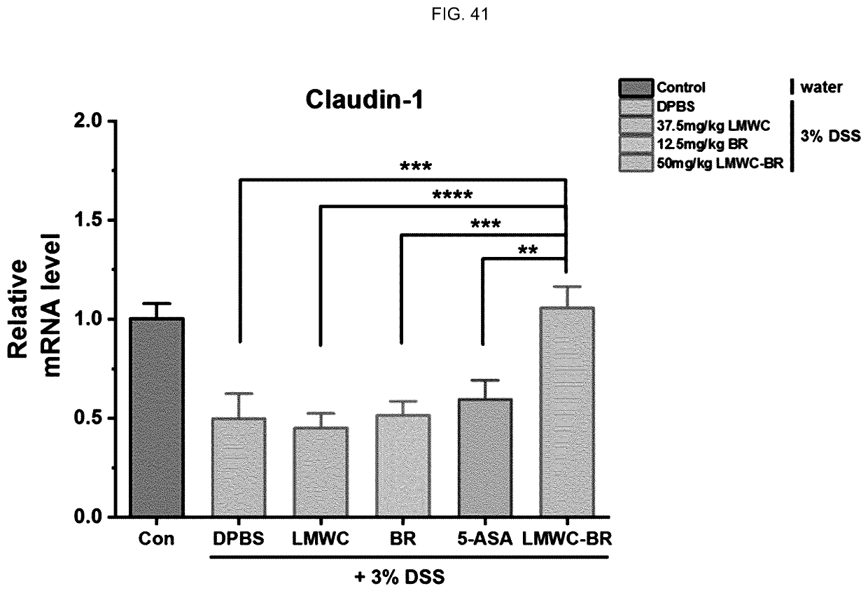

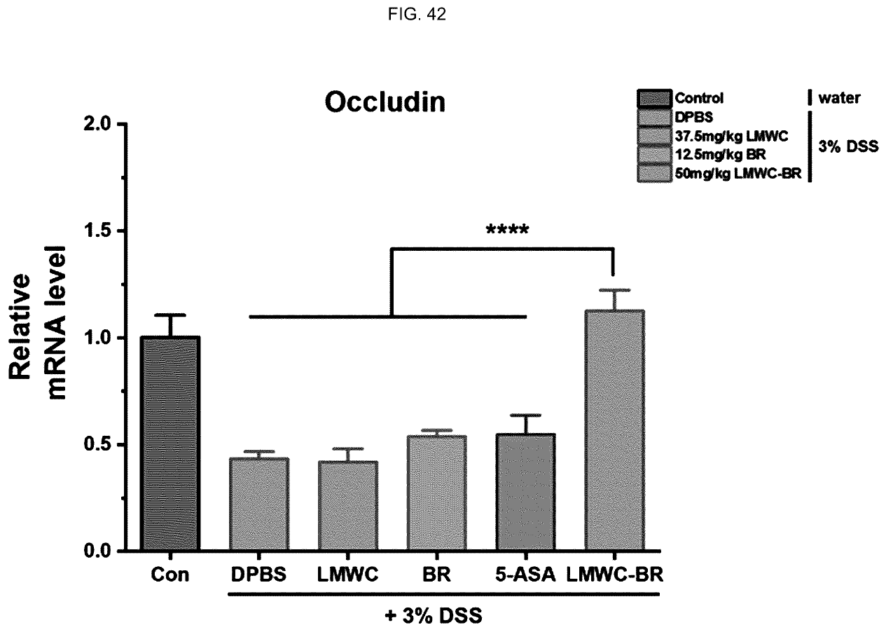

FIGS. 40 to 42 show the expression levels of ZO-1, Claudin-1, and Occludin-1, which are indicators of intestinal damage in inflammatory bowel disease for each mouse group in Example 7.

As shown in FIG. 32, the body weight change was smallest in the group administered the 50 mg/kg chitosan-bilirubin conjugate of the present disclosure.

As shown in FIG. 33, in the group administered the 50 mg/kg chitosan-bilirubin conjugate of the present disclosure, the disease activity index was lowest, and the disease activity index at the end of the experiment was similar to that of the normal group.

As shown in FIG. 34, the colon length was longest in the group administered the 50 mg/kg chitosan-bilirubin conjugate of the present disclosure, wherein the colon length was close to that of the normal group.

The present inventors identified, in Example 7, the inflammation and abnormality of the other organs due to the influence of intestinal inflammation. As the symptoms of inflammatory bowel disease were aggravated, systemic inflammations may be caused through the release of inflammatory cytokines in the blood, and therefore, an effective anti-inflammatory medicine was thought to inhibit the release of inflammatory substances in the spleen, liver, kidney, or the like to prevent the inflammations, and thus this was investigated.

As shown in FIG. 35, the spleen was removed for each mouse group and measured for the appearance and weight, and as a result, the spleen size was decreased in the group having a bowel disease induced by administering DSS. However, the spleen weight was high in the groups fed with chitosan-bilirubin of the present disclosure and 5-ASA compared with the other groups.

As the inflammation progresses, the spleen may be enlarged due to the invasion of various immune cells, but severe inflammation may cause rather spleen atrophy resulting in contraction, and considering this, it was considered from the above results that spleen atrophy caused by severe inflammation was alleviated through inflammation relief.

The present inventors also identified in Example 7 that the abnormality of liver inflammation scores due to the influence of intestinal inflammation. As shown in FIG. 36, as a result of measuring blood ALT and blood AST for each mouse group, the levels were lowest in the group administered the chitosan-bilirubin of the present disclosure.

The present inventors also identified in Example 7 that the abnormality of renal functions due to the influence of intestinal inflammation. As shown in FIG. 37, as a result of measuring blood creatine and blood BUN for each mouse group, the levels were lowest in the group administered the chitosan-bilirubin of the present disclosure.

The present inventors also identified, in Example 7, the abnormality of the blood inflammatory cytokines IL-6 and TNF-alpha due to the influence of intestinal inflammation. As shown in FIGS. 38 and 39, the levels of the inflammatory cytokines IL-6 and TNF-alpha were lowest in the group administered the chitosan-bilirubin of the present disclosure.

It could be seen from the above results that the chitosan-bilirubin conjugate of the present disclosure had an effect of alleviating systemic inflammation due to the intestinal inflammation.

In addition, considering that the expression of intestinal tight junction-related genes was low due to the damaged intestinal structure in the inflammatory bowel disease in Example 7, the present inventors measured the mRNA expression levels of related genes ZO-1, Claudin-1, and Occludin-1. As shown in FIGS. 40 to 42, the mRNA expression levels of the intestinal tight junction-related genes ZO-1, Claudin-1, and Occludin-1 was highest in the group administered the chitosan-bilirubin of the present disclosure.

It could be seen from the above results that the chitosan-bilirubin conjugate-containing nanoparticles of the present disclosure had a better treatment effect on intestinal inflammation than 5-ASA, a commercialized drug.

Although not shown in the results of the present example, as a result of analyzing microbiome diversity and 16s rRNA after the collection of feces on days 0, 4, and 8, only the group administered the chitosan-bilirubin conjugate of the present disclosure showed a microbiome distribution similar to that of normal mice, indicating that the chitosan-bilirubin conjugate-containing nanoparticles of the present disclosure had an unexpected effect of normalizing the microbiome distribution in the inflammatory bowel disease.

### Example 8: Preparation of Hyaluronic Acid-Bilirubin Conjugate and Comparison of Efficacy with Inventive Low Molecular Weight Chitosan-Bilirubin Conjugate in Inflammatory Bowel Disease Model

In order to compare the efficacy of the low molecular weight chitosan-bilirubin conjugate of the present disclosure and previously known bilirubin particles, the present inventors synthesized a hyaluronic acid-bilirubin conjugate and used nanoparticles manufactured therefrom to perform in vivo efficacy evaluation.

8-1. Synthesis and Characterization of Hyaluronic Acid-Bilirubin Conjugate

For the synthesis of a hyaluronic acid-bilirubin conjugate, 10 kDa hyaluronic acid having the same molecular weight as the low molecular weight chitosan in the present experiment was used, and synthesis conditions and reagents were the same as those in the preparation of the chitosan-bilirubin conjugate. For the optimization of a ratio, conjugates were prepared while two molar ratios of hyaluronic acid to bilirubin were set to be 1:1 and 1:2, and then the contents of bilirubin of the conjugates were compared.

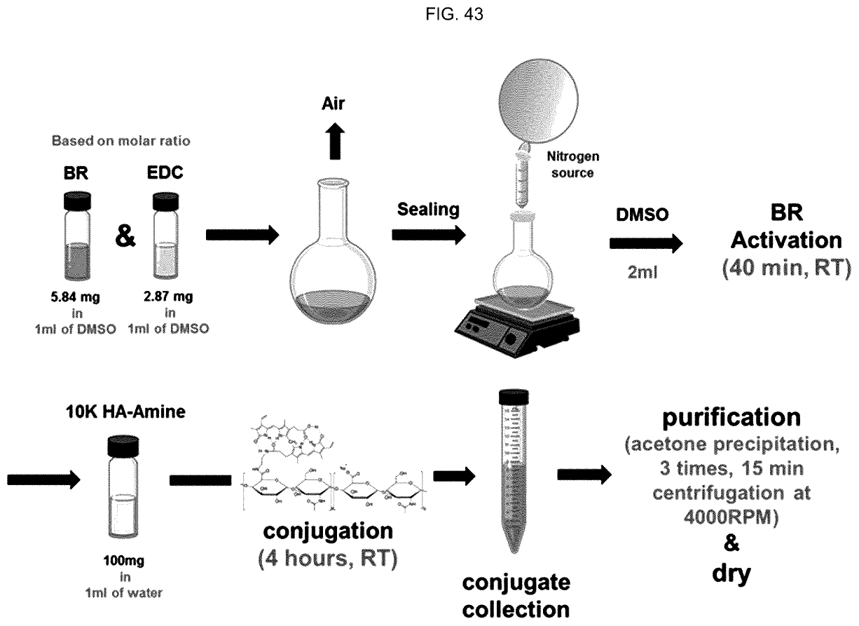

Specifically, for the reaction of the carboxyl group of bilirubin with hyaluronic acid, 10% of an amine group was introduced into 10 kDa hyaluronic acid and used for the reaction. The preparation process for the hyaluronic acid-bilirubin conjugate is shown in FIG. 43.

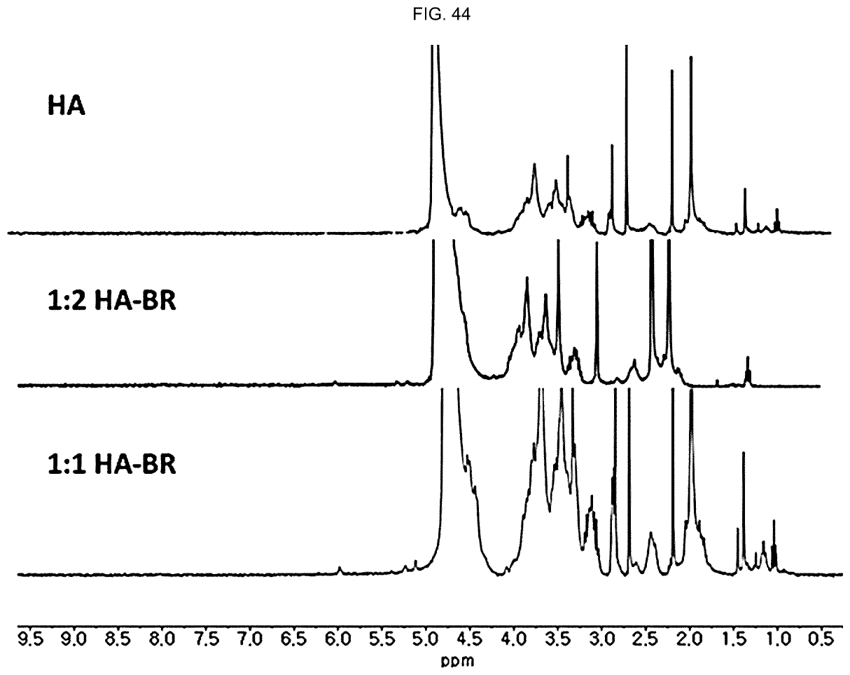

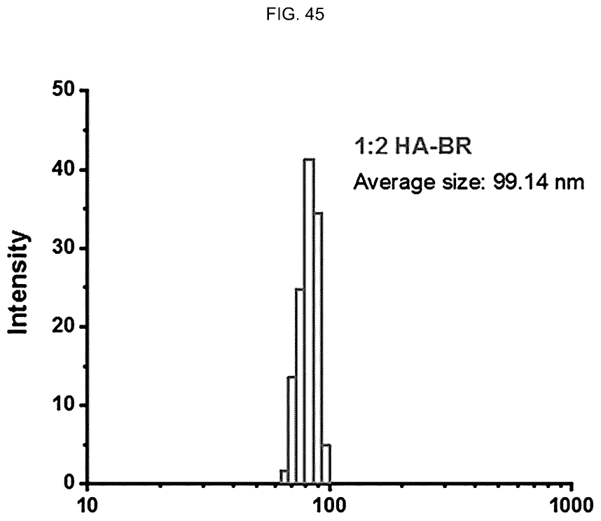

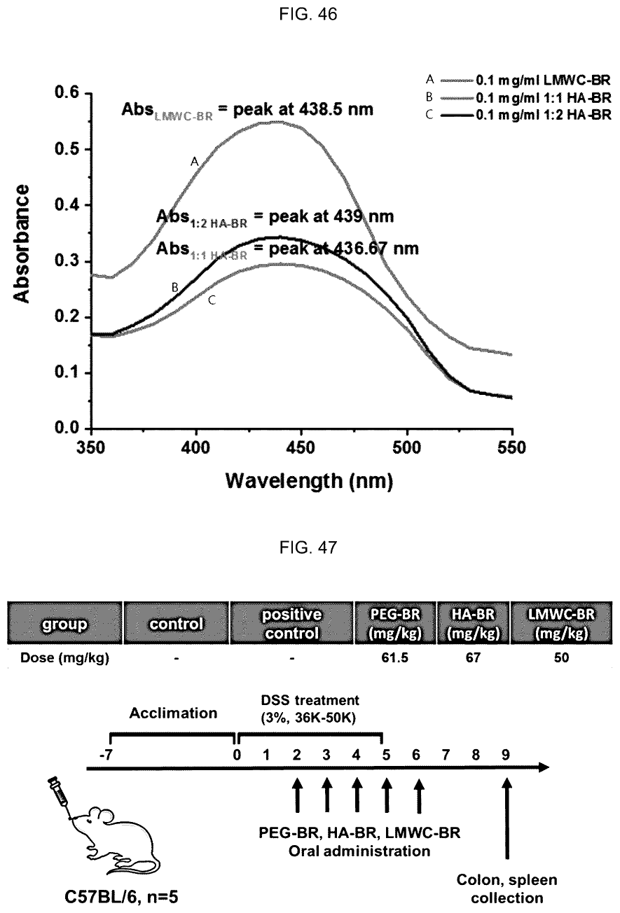

As shown in FIG. 43, the carboxyl group of bilirubin was activated by EDC for 40 minutes, and reacted with 10 kDa hyaluronic acid with an amine group introduced thereinto for 4 hours, and then the reaction mixture was purified by acetone addition. The purified reaction mixture was dried to obtain a solid hyaluronic acid-bilirubin conjugate. The synthesis of the conjugate was confirmed through H1-NMR data (FIG. 44), and after the conjugate was manufactured into nanoparticles in an aqueous solvent, the size of the nanoparticles composed of the hyaluronic acid-bilirubin conjugate and manufactured in an aqueous solvent was determined by DLS measurement (FIG. 45). In addition, the content (weight %) of bilirubin was calculated through the comparison with the UV absorbance of bilirubin (FIG. 46 and Table 3).

TABLE 3
Content of bilirubin in conjugate (wt %)

| Group | PEG-BR | 1:1 HA-BR | 1:2 HA-BR | LMWC-BR |
|---|---|---|---|---|
| BR weight % in 1 | 20.8 | 17.5 | 19.1 | 25.6 |
| mg conjugate | | | | |

As shown in Table 3, the HA-BR conjugate (prepared at a molar ratio of 1:2)-containing nanoparticles were considered to have a similar content of bilirubin to the nanoparticles containing the chitosan-bilirubin conjugate of the present disclosure, and thus further experiments were conducted using 1:2 HR-BR. PEG-BR on Table 2 means a PEGylated bilirubin conjugate disclosed in Korean Patent Publication No. 10-1681299.

8-2. Comparison of Efficacy of Conventional Bilirubin Conjugate (PEG-BR and HA-BR)-Containing Nanoparticles and Chitosan-Bilirubin Conjugate-Containing Particles in Mouse Inflammatory Bowel Disease Model

The in vivo efficacy of the PEGylated bilirubin conjugate (PEG-BR) disclosed in Korean Patent No. 10-1681299, the hyaluronic acid-bilirubin conjugate (HA-BR) prepared in Example 8-1, and the chitosan-bilirubin conjugate (LMWC) of the present disclosure was compared as follows.

FIG. 47 is a schematic diagram of the experimental method of Example 8-2 of the present disclosure.

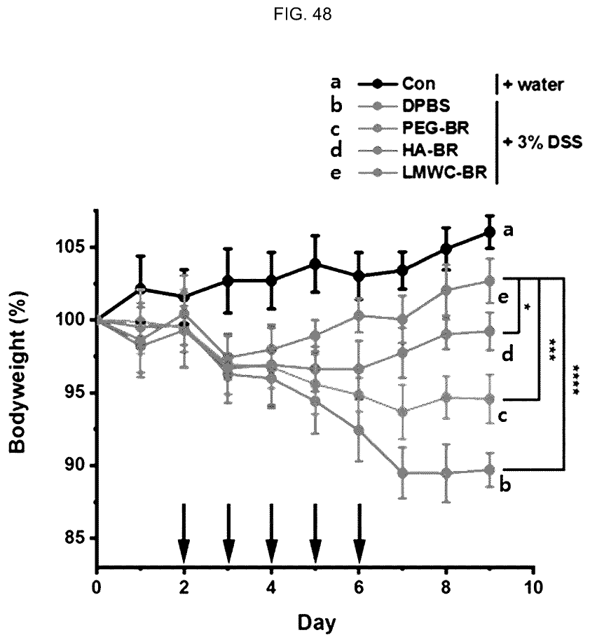

FIG. 48 shows the body weight change for each mouse group in Example 8-2.

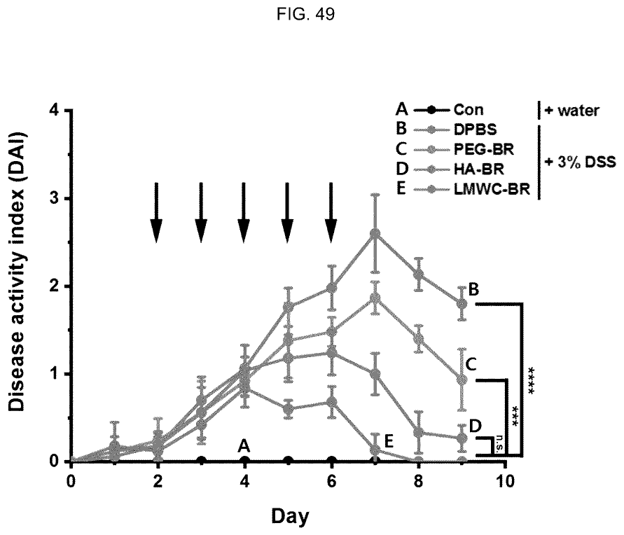

FIG. 49 shows the disease activity index (DAI) for each mouse group in Example 8-2.

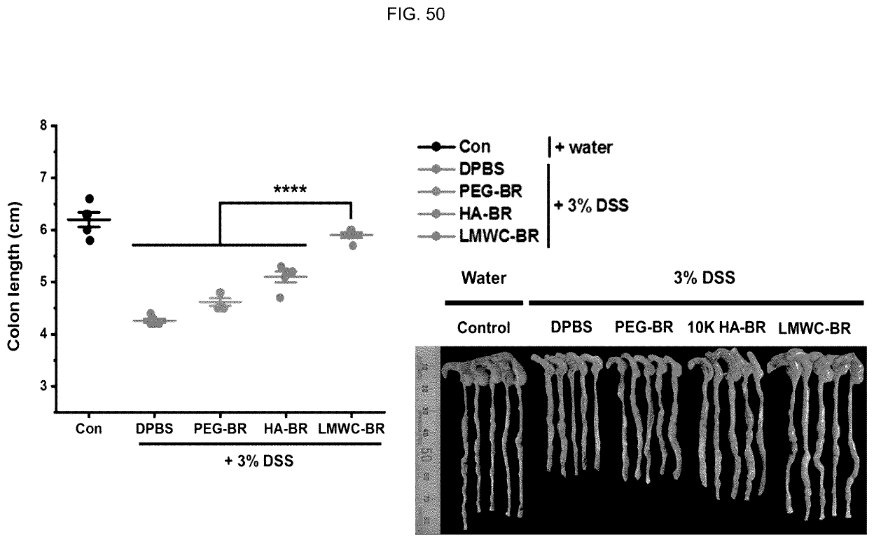

FIG. 50 shows the colon length for each mouse group in Example 8-2.

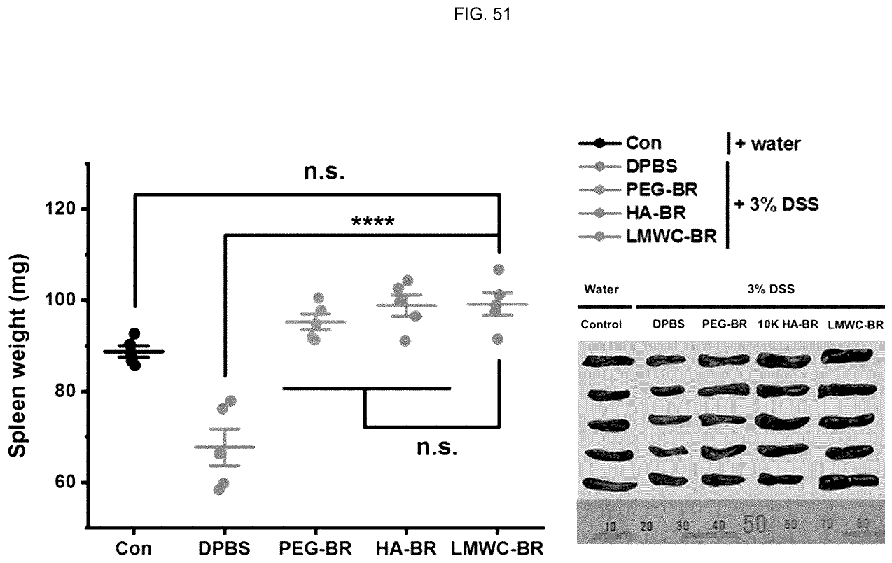

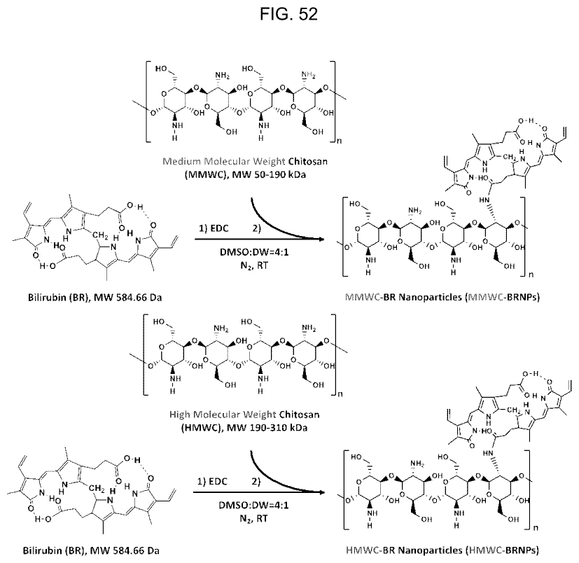

FIG. 51 shows the spleen weight for each mouse group in Example 8-2.

As shown in FIG. 48, the body weight change was smallest in the group administered the 50 mg/kg chitosan-bilirubin conjugate of the present disclosure.

As shown in FIG. 49, in the group administered the 50 mg/kg chitosan-bilirubin conjugate of the present disclosure, the disease activity index was lowest, and the disease activity index at the end of the experiment was similar to that of the normal group.

As shown in FIG. 50, the colon length was longest in the group administered the 50 mg/kg chitosan-bilirubin conjugate of the present disclosure, wherein the colon length was close to that of the normal group.

As shown in FIG. 51, the spleen was removed for each mouse group and measured for the appearance and weight, and as a result, the spleen weight was higher in the group administered the chitosan-bilirubin of the present disclosure than in the other groups.

It could be seen from the above results that the nanoparticles manufactured of the chitosan-bilirubin conjugate of the present disclosure showed a significantly excellent effect on the treatment of inflammation compared with nanoparticles manufactured of conventional bilirubin conjugates in a mouse inflammatory bowel disease model.

### Example 9: Synthesis and Characterization of Medium Molecular Weight Chitosan Conjugated Bilirubin Nanoparticle (MMWC-BRNP) and High Molecular Weight Chitosan Conjugated Bilirubin Nanoparticle (HMWC-BRNP)

9-1. Synthesis of MMWC-BRNP and HMWC-BRNP

MMWC-BRNPs and HMWC-BRNPs were synthesized in the same manner as LMWC-BRNPs. Briefly, the carboxylic group of bilirubin (BR) was activated by dissolving 15 mg of BR and 7 mg of N-(3-dimethylaminopropyl)-N′-ethyl carbodiimide hydrochloride (EDC) in 4 mL of dimethyl sulfoxide (DMSO) for 30 minutes at RT under nitrogen. Then, 1 mL of deionized (DI) water containing 100 mg of MMWC or HMWC was added to the mixture, and the reaction was allowed to proceed for 4 hours. Any air remaining in the flask was removed using a vacuum pump and air entry was prevented by tightly sealing the entrance with parafilm. The reaction mixture was then precipitated with acetone three times by centrifugation at 500×g for 10 minutes at 4° C. and lyophilized, yielding the solid form of MMWC-BRNPs or HMWC-BRNPs.

9-2. Hydrodynamic Size and Zeta Potential Measurement

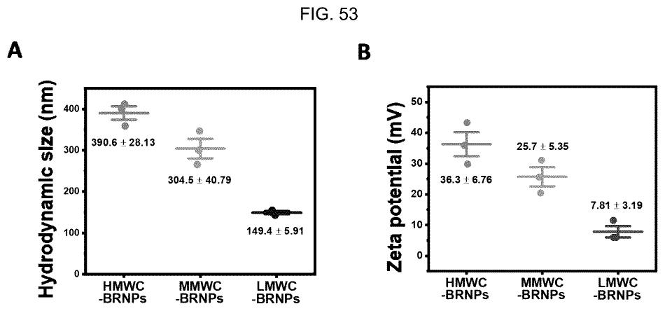

Hydrodynamic size (A) and zeta potential (B) of synthesized MMWC-BRNPs and HMWC-BRNPs were measured by dynamic light scattering (DLS). The results are shown in FIG. 53.

As shown in FIG. 53, the hydrodynamic size of HMWC-BRNPs and MMWC-BRNPs, measured by DLS, was substantially increased to −304 nm and −390 nm, respectively, compared with that of LMWC-BRNPs (˜150 nm). The surface charge of HMWC-BRNPs and MMWC-BRNPs also increased to more positive values-36.3±6.76 and 25.7±5.35 mV, respectively—compared with that of LMWC-BRNPs (7.81±3.19 mV).

9-3. Amount of BR in LMWC-BRNPs

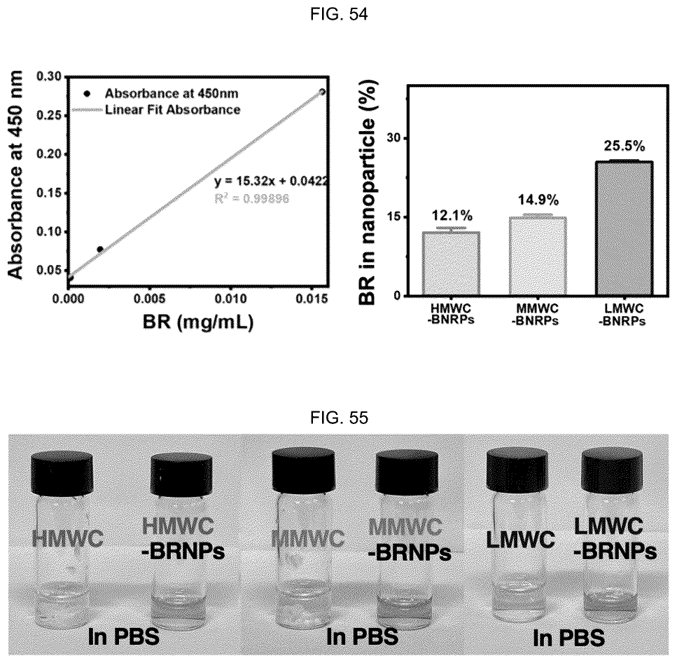

The amount of BR in LMWC-BRNPs was determined by measuring UV/vis absorbance at a wavelength of 450 nm and quantified by reference to a standard curve prepared from known concentrations of BR (0-0.1 mg/mL). The results are shown in FIG. 54 and Table 4.

TABLE 4
Measurement results of BR in LMWC-BRNPs
LMWC-BRNPs

| Concentration of LMWC-BRNPs | 0.125 | mg/ml |
|---|---|---|
| Absorbance intensity (λ = 450 nm) | 0.532 | |
| Concentration of BR | 0.025 | mg/ml |
| from standard curve | | |
| Amount of BR in 1 mg | 0.256 | mg |
| LMWC-BRNPs | | |

| | |
|---|---|
| BR content (%) | 25.6% |

As shown in FIG. 54 and Table 4, The percentage (%) of BR in MMWC-BRNPs and HMWC-BRNPs was calculated to be ˜14.9% and ˜12.1% respectively, which were less than the percentage of BR in LMWC-BRNPs.

9-4. Solubility of LMWC-, MMWC-, and HMWC-BRNPs

The solubility of LMWC-, MMWC-, and HMWC-BRNPs was shown in FIG. 55. As shown in FIG. 55, MMWC-BRNPs and HMWC-BRNPs showed less water solubility compared to the LMWC-BRNPs.

9-5. Mucoadhesive Properties of Nanoparticles

The mucoadhesive properties of each nanoparticle were characterized based on mucin binding efficiency (%). A mucin stock solution was prepared by dissolving 4 mg of type II mucin in 1 mL of DI water and magnetically stirring overnight at RT. Thereafter, the stock solution was filtered using a syringe filter with a pore size of 0.8 μm and kept at 4° C. Mucin standards (A) were prepared by serially diluting the stock solution. PEG-BRNPs, 10K HA-BRNPs, and LMWC-BRNPs were dissolved in DI water and added into a new mucin solution, after which each solution mixture was magnetically stirred at 600 rpm for 15 minutes and incubated for 1 hour at 37° C. After incubation, each solution was centrifuged at 40,000×g for 30 minutes. Both pellet and supernatant were used for subsequent analyses.

Mucin binding efficiency (%) was then analyzed by performing colorimetric periodic acid/Schiff's staining using a previously reported protocol. The coloring reagent was prepared in two steps: 1) Preparation of periodic acid reagent: 10 μL of 50% periodic acid solution (dissolved in DI water) was added to 7 mL of 7% acetic acid solution and stored at 4° C. before use. 2. Preparation of Schiff's reagent: 100 mL of 1% basic fuchsin aqueous solution was added to 20 mL of 1N HCL and incubated for 2 hours in the dark at 3TC. Before coloration, 50 mg of sodium metabisulfite was added to 3 mL of the incubated Schiff's reagent. Both reagents were cooled to RT before use. For colorization, 600 μL of the supernatant of each nanoparticle-mixed mucin solution and standard were first added to 180 μL of periodic acid reagent and incubated at 37° C. for 1 hour. Then, 60 μL of Schiff's reagent containing sodium metabisulfite was added to each mixture and incubated for 30 minutes. Changes in color during this incubation were detected by measuring UV/vis absorbance at 565 nm with a fluorescence microplate reader.

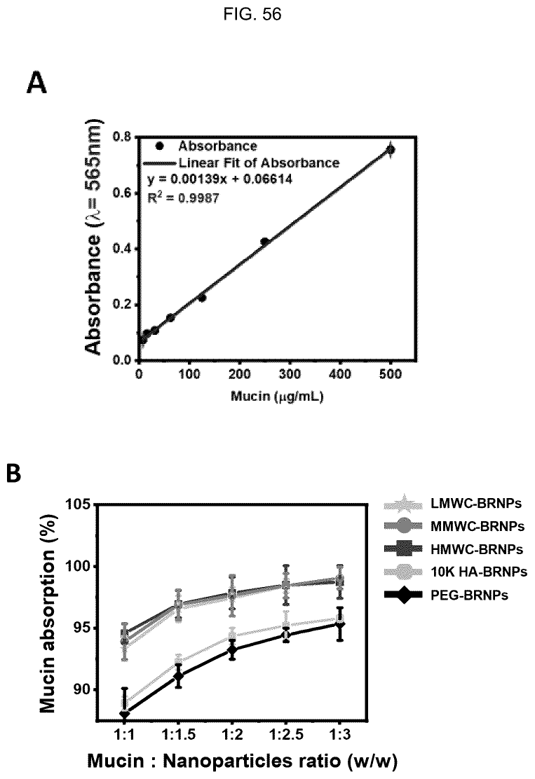

The results are shown in FIG. 56.

As shown in FIG. 56, both MMWC-BRNPs and HMWC-BRNPs showed similar mucoadhesive properties with LMWC-BRNPs. However, only LMWC-BRNPs bared both hydrophilicity and strong mucoadhesiveness, which are crucial for effective oral absorption.

### Example 10: Therapeutic Efficacy of Chitosan Conjugated Bilirubin Nanoparticle in DSS-Colitis Model According to Molecular Weight of Chitosan

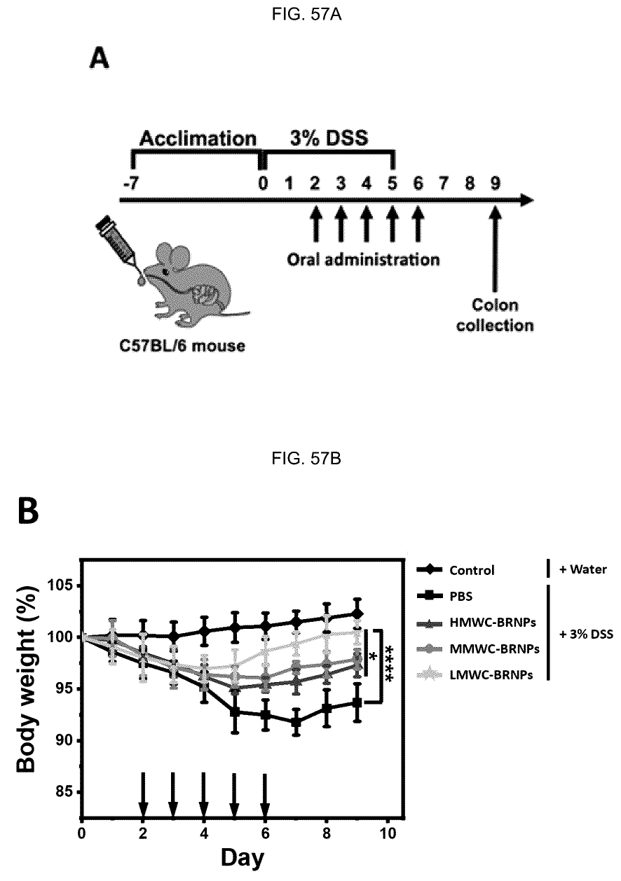

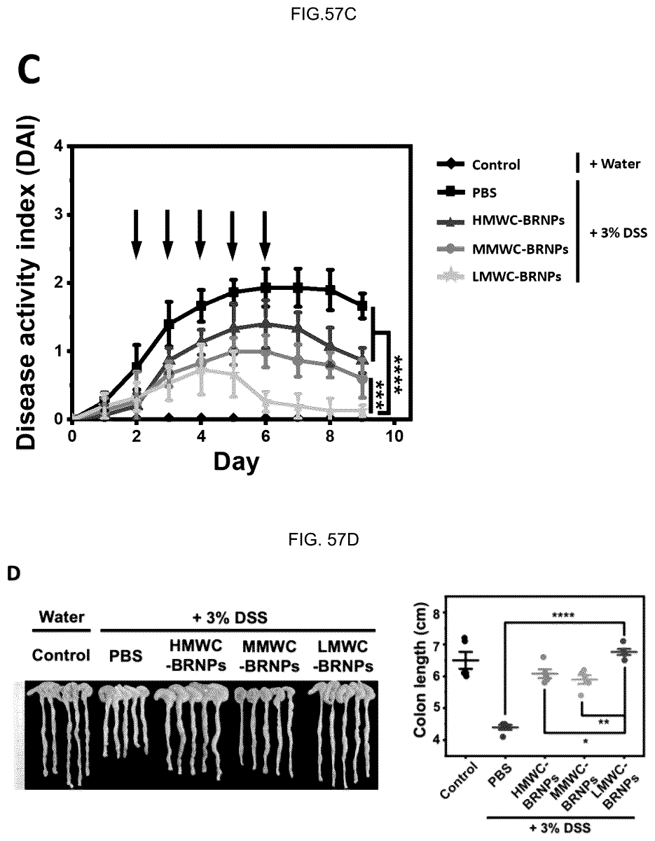

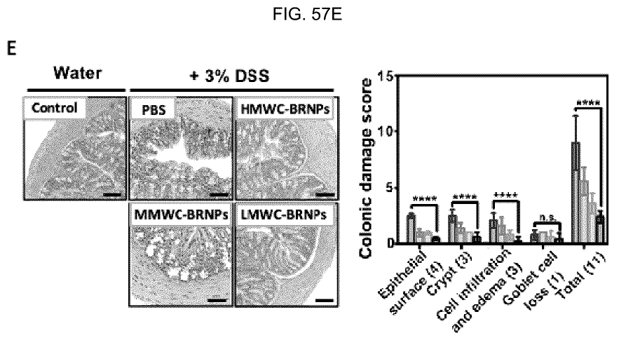

Therapeutic efficacy of chitosan conjugated bilirubin nanoparticle was analyzed in DSS-colitis model according to molecular weight of chitosan. FIG. 57A shows the scheme of the test. The results were shown in FIGS. 57A to 57E.

Among three chitosan-based BRNPs, LMWC-BRNPs at a dose of BR equivalent to that of the other two BRNPs exhibited the best efficacy in all therapeutic assessments in DSS-colitis model including bodyweight (FIG. 57B), disease activity index (FIG. 57C), colon length (FIG. 57D), and histology (FIG. 57E), indicating the importance of the molecular weight of chitosan as a carrier of BR.

Taken together, these results indicate that LMWC-BRNPs are highly effective in treating colitis, demonstrating the molecular weight of chitosan as a carrier.

### Example 11: Therapeutic Efficacy of Chitosan Conjugated Bilirubin Nanoparticle in Murine NASH Model

11-1. Body Weight, Liver and Spleen Weight of MCD-Supplied Mice

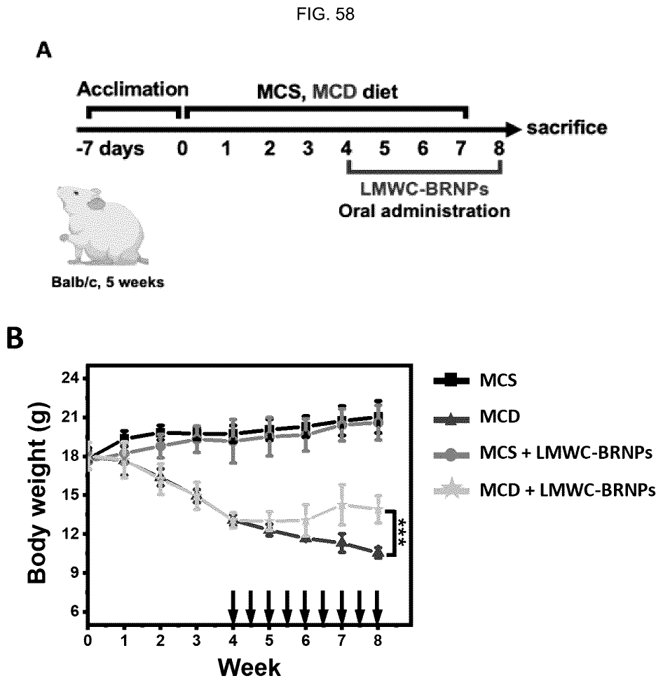

The Murine NASH model was induced by a methionine/choline-deficient (MCD) diet which contains high sucrose and fat but lacks methionine and choline. Since these are responsible for hepatic fat regulation, the liver undergoes excessive fat accumulation and oxidative stress-induced damage. 5-weeks-aged Balb/c mice were fed with an MCD diet for 8 weeks, starting from the 7 days of acclimation (FIG. 58 (A)). Normal mice were supplied with a methionine/choline-sufficient (MCS) diet. From week 4 to week 8, 80 mg/kg LMWC-BRNPs were orally administered daily into each mouse of the normal and MCD group. During the diet, disease progression was tracked by checking the bodyweight change. On week 8, mice were sacrificed to collect livers and blood. Collected liver and blood were used for further analysis, including hepatic cytokine expression and serology of liver damage markers.

The results were shown in FIGS. 58 to 65.

As the disease progressed, the bodyweight of MCD diet-supplied mice significantly decreased. Yet, LMWC-BRNPs administrated mice showed less decline in body weight, showing the possibility of remission (FIG. 58 (B)).

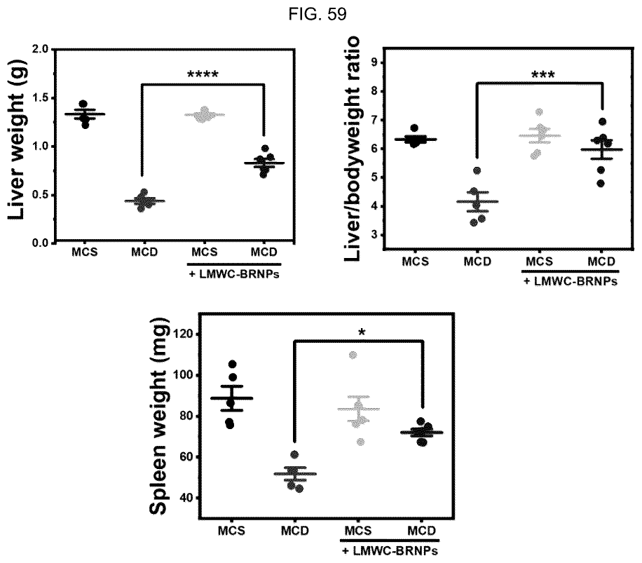

After sacrificing the mice, the collected liver was weighted to compare the liver weight loss among groups. Additionally, liver-to-bodyweight ratio and spleen weight were measured. As a result, LMWC-BRNPs administrated mice showed notable preventive effects on decrease in liver and spleen weight compared to the MCD diet mice (FIG. 59).

11-2. Anti-Inflammatory Effect of LMWC-BRNPs in NASH Model

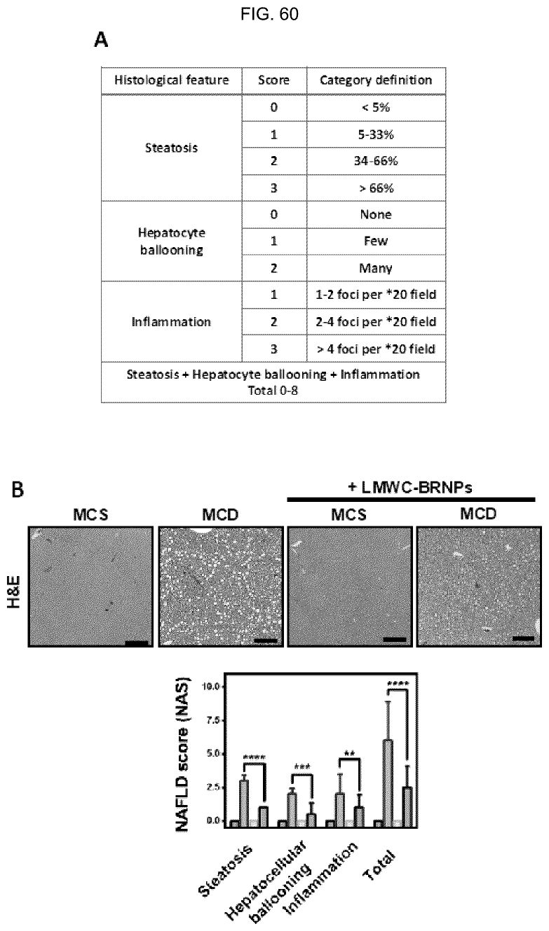

To examine the anti-inflammatory effect of LMWC-BRNPs in the NASH model, liver samples were fixed and prepared for histological assessment. Briefly, hepatic inflammation and steatosis were evaluated based on the H&E staining, according to the non-alcoholic fatty liver disease activity score (NAS, FIG. 60 (A), Clin. Exp. Hepatol. 2018; 4, 3: 165-174.). Among the MCD diet group, LMWC-BRNPs administered mice expressed significantly low levels of hepatic inflammation and steatosis, suggesting excellent therapeutic efficacy in disease progress (FIG. 60 (B)).

11-3. Effects on Liver Damage Markers and Fat Accumulation

Additionally, collected serum samples were evaluated for liver damage markers.

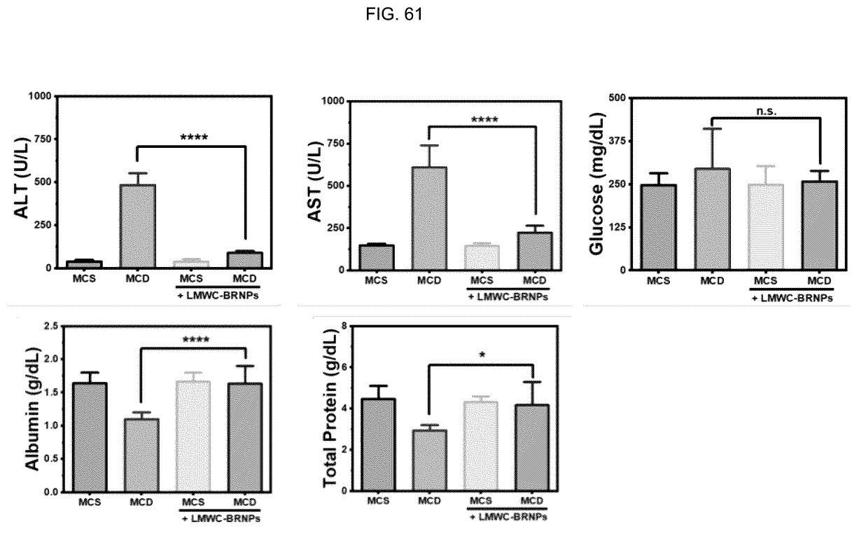

As shown in FIG. 61, AST, ALT, and glucose levels significantly increased in the MCD group. In contrast, all marker was significantly downregulated by the LMWC-BRNPs administration. Similarly, serum albumin and total protein of LMWC-BRNPs treated mice were close to the MCS diet group.

Furthermore, the mice were examined for fat accumulation in detail.

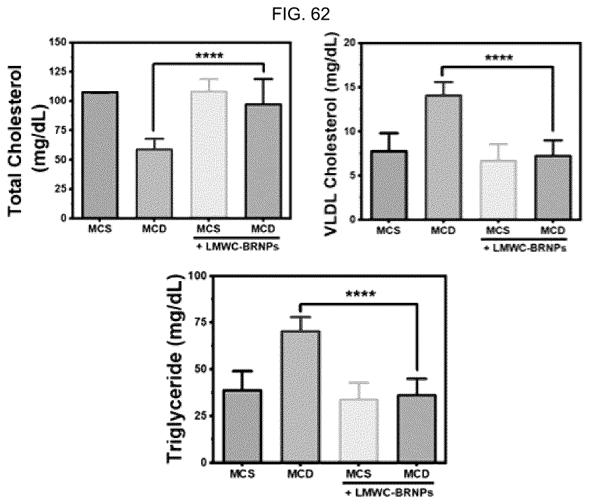

As shown in FIG. 62, serum total cholesterol, VLDL cholesterol, and triglyceride were measured. Remarkably, the serum total cholesterol level of LMWC-BRNPs administrated mice was higher than that of MCD diet mice. Simultaneously, the level of VLDL was significantly low in the LMWC-BRNPs administrated group, implying the modulatory effect on cholesterol accumulation. Similarly, serum triglyceride levels decreased in the treatment group.

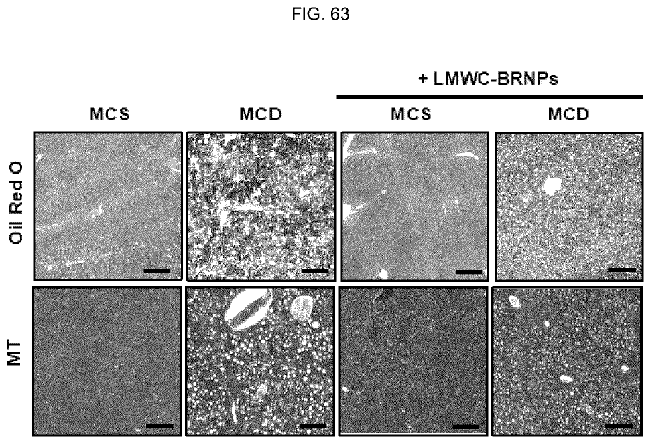

Fat accumulation of the liver observed by Oil Red O staining and Masson's Trichrome (MT) staining. The results were shown in FIG. 63.

Oil Red O staining, which visualizes the hepatic triglyceride, showed that LMWC-BRNPs treated mice had fewer Oil Red O-positive cells. Furthermore, Masson's Trichrome (MT) staining revealed that LMWC-BRNPs significantly prevent hepatic fibrosis.

These results suggest that oral administration of LMWC-BRNPs could lower the abnormal fat accumulation induced by the MCD diet and thus showed robust therapeutic efficacy in the murine NASH model.

11-4. Cytokine and MPO Activity Analysis

In addition to the fat-modulating effect, the hepatic pro-inflammatory cytokines and MPO activity were analyzed.

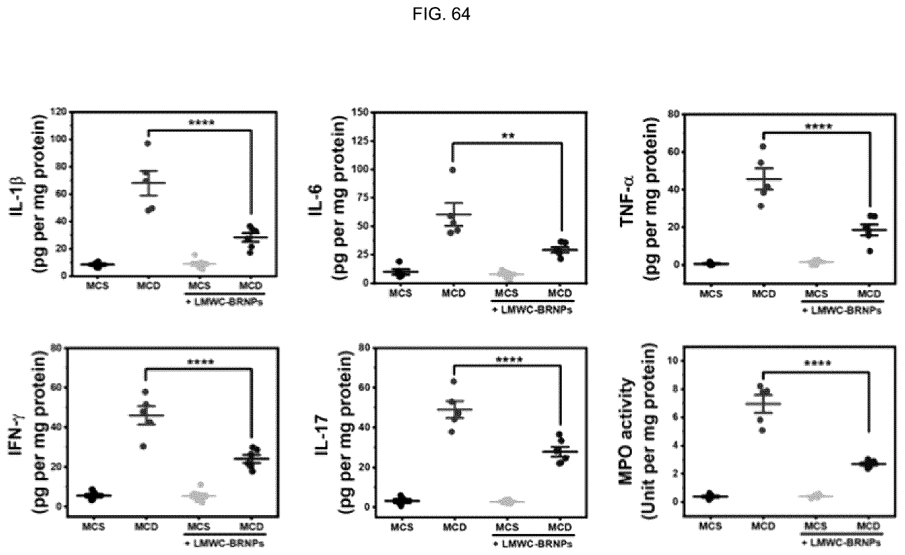

As shown in FIG. 64, the LMWC-BRNPs group significantly downregulated the hepatic pro-inflammatory cytokines (IL-113, IL-6, TNF-α, IFN-γ, IL-17) and MPO activity of MCD diet-supplied mice, signifying the strong anti-inflammatory effect of LMWC-BRNPs.

11-5. Serum Bilirubin Analysis

Serum bilirubin analysis was performed to measure the delivery efficiency of LMWC-BRNPs and concentration of the LMWC-BRNP in serum after administration.

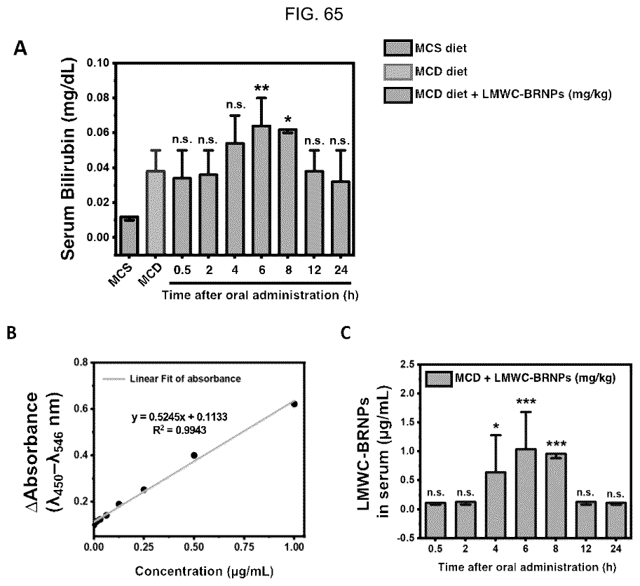

As shown in FIG. 65, MCD-diet mice showed a significant increase in serum bilirubin level after 6-8 hours of LMWC-BRNPs (100 mg/kg) administration (FIG. 65 (A)), emphasizing the effective delivery of LMWC-BRNPs through the gut to the liver axis. This measurement was further used to check the serum LMWC-BRNPs level.

Based on the UV/vis absorbance of bilirubin dissolved in serum, concentration of the LMWC-BRNPs was calculated from that of bilirubin in the serum (FIGS. 65 (B) and (C)).

What is claimed is:

1. A conjugate comprising a chitosan and bilirubin, the chitosan being linked to bilirubin.

2. The conjugate of claim 1, wherein the chitosan is linked to bilirubin via a covalent bond.

3. The conjugate of claim 1, wherein the chitosan is linked to bilirubin via an amide linkage.

4. The conjugate of claim 1, wherein the linking is made via an amide linkage between a carboxyl group of bilirubin and an amine group of the chitosan.

5. The conjugate of claim 1, wherein the chitosan has a molecular weight of 3 kDa to 30 kDa.

6. The conjugate of claim 1, wherein the conjugate has a st Formula 1 below: 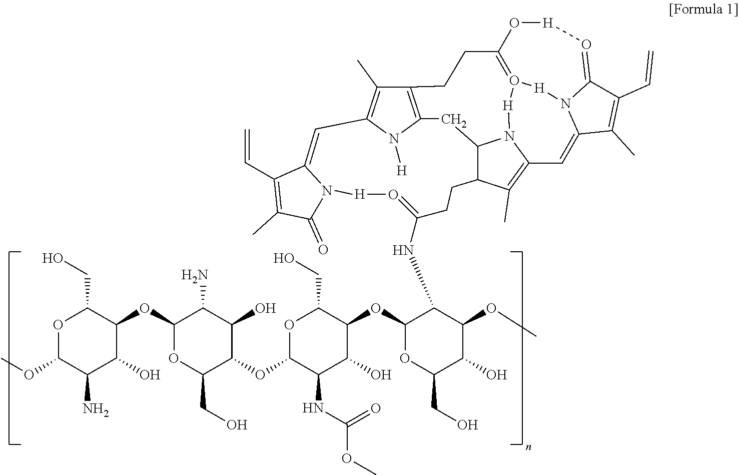
where n is an integer of 4 to 45.

7. Particles comprising the conjugate of claim 1.

8. The particles of claim 7, wherein the particles are formed by self-assembly of a plurality of conjugates in an aqueous solution.

9. The particles of claim 7, wherein the particles have a hydrodynamic diameter of 10 to 5,000 nm as measured by dynamic light scattering (DSL).

10. A method for treating inflammatory disease, comprising administering a composition to a subject in need of thereof,
wherein the composition comprises i) a conjugate comprising a chitosan and bilirubin, the chitosan being linked to bilirubin, ii) particles comprising the conjugate, or iii) a combination of the conjugate and the particles; and a pharmaceutically acceptable carrier.

11. The method of claim 10, wherein the composition is administered orally.

12. The method of claim 10, wherein the inflammatory disease is inflammatory bowel disease.

13. The method of claim 12, wherein the inflammatory bowel disease is selected from the group consisting of ulcerative colitis, Crohn's disease, intestinal Behcet's disease, indeterminate colitis, bacterial enteritis, viral enteritis, amoebic enteritis, hemorrhagic rectal ulcer, leaky gut syndrome, ischemic colitis, and tuberculous enteritis.

14. The method of claim 10, wherein the inflammatory disease is selected from the group consisting of non-alcoholic steatohepatitis, pneumonia, pulmonary fibrosis, nephritis, kidney failure, cystitis, Sjogren's syndrome, multiple sclerosis, asthma, atherosclerosis, myocardial infarction, pancreatitis, diabetes, psoriasis, osteoporosis, arthritis, osteoarthritis, rheumatoid arthritis, systemic inflammatory syndrome, sepsis, and dementia.

15. The method of claim 10, wherein the chitosan in the conjugate is linked to bilirubin via a covalent bond.

16. The method of claim 10, wherein the chitosan in the conjugate is linked to bilirubin via an amide linkage.

17. The method of claim 10, wherein the linking between chitosan and bilirubin is made via an amide linkage between a carboxyl group of bilirubin and an amine group of the chitosan.

18. The method of claim 10, wherein the chitosan has a molecular weight of 3 kDa to 30 kDa.

19. The method of claim 10, wherein the conjugate has a structure of Formula 1 below: 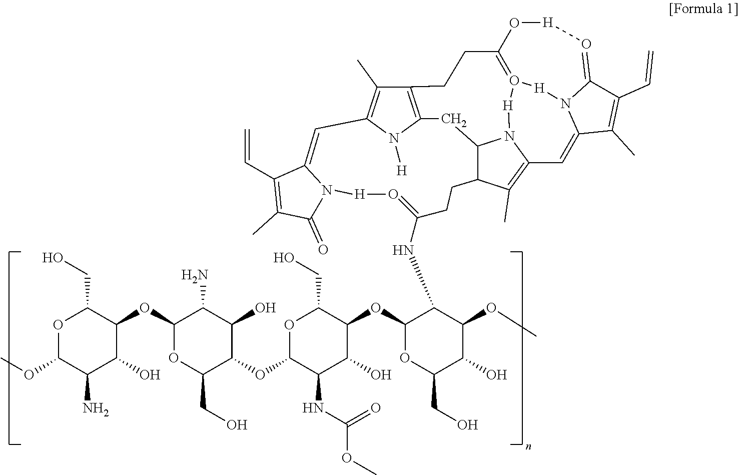
where n is an integer of 4 to 45.

20. The method of claim 10, wherein the particles have a hydrodynamic diameter of 10 to 5,000 nm as measured by dynamic light scattering (DSL).
